(19) United States
(12) Patent Application Publication
(10) Pub. No.: US 2024/0013878 A1
(43) Pub. Date: Jan. 11, 2024

(54) Machine learning methods for classification and clinical detection of Bevacizumab responsive glioblastoma subtypes based on microRNA (miRNA) biomarkers

(71) Applicant: Jian Shi, San Francisco, CA (US)

(72) Inventor: Jian Shi, San Francisco, CA (US)

(21) Appl. No.: 18/347,547

(22) Filed: Jul. 5, 2023

Related U.S. Application Data
(60) Provisional application No. 63/367,715, filed on Jul. 5, 2022.

(51) Int. Cl. G16H 20/10; C12Q 1/686

(52) U.S. Cl. CPC G16H 20/10; C12Q 1/686

(57) ABSTRACT

The present invention relates to methods for classification, detection, and diagnosis of glioblastoma multiforme (GBM) bevacizumab (BVZ)-responsive and non-responsive subtypes based on selection of a machine learning algorithm and its combination with differential expression DE of microRNAs (miRNAs) and messenger RNAs (mRNAs), particularly a panel of a group of miRNAs to be used as biomarkers, along with clinical characteristics, and related functional pathways for precise diagnosis and further treatment of GBM patients. The present invention discloses that based on miR-21 and miR-10b expression z-scores, approximately 30% of GBM patients were classified as having the BVZ-responsive GBM subtype. The present invention provides that BVZ GBM subtypes can be classified and detected by a combination of SVM classifiers and miRNA panels in existing tissue GBM datasets. The present invention further provides that with certain modifications, the classifier as disclosed in the present invention may be used for the classification and detection of BVZ GBM subtypes for clinical use. Additionally, as one such clinical use, the present invention provides methods for prescreening of GBM patients to prevent aging-related side effects in BVZ-non-responsive subtype of the GBM patients after BVZ treatment, in addition to the side effects of healing complications caused by BVZ treatment.

### PRIORITY

This application claims the benefit of priority to U.S. provisional application Ser. No. 63/367,715 filed on Jul. 5, 2022.

### FIELD OF THE INVENTION

The present invention relates generally to the classification and detection of glioblastoma multiforme (GBM) bevacizumab (BVZ)-responsive subtypes for future clinical use. More specifically, the present invention relates to the classification and detection of GBM BVZ subtypes based on selection of a machine learning algorithm and its combination with differential expression (DE) of microRNAs (miRNAs) and messenger RNAs (mRNAs), clinical characteristics, and related functional pathways for precise treatment of GBM patients.

### BACKGROUND OF THE INVENTION

Glioblastoma multiforme (GBM) is the most common and the most lethal malignant brain tumor. Despite decades of intensive efforts to optimize the treatment of glioblastoma, the outcomes of GBM patients are still disappointing, with a median life expectancy of ˜15 months after diagnosis. The main reason for this poor outcome is that despite extensive investigations of the pathogenesis of GBM, the various genetic risk factors involved in the development of the disease and their regulatory pathways remain poorly understood remain poorly understood. However, because of substantial large-scale genome sequencing studies, knowledge of GBM epigenetics and genetics has been growing very fast and going very far. Facing the vast genetic information of brain cancer, researchers have to work harder to understand the true meaning of genetics under the surface, and to more accurately classify them through that information rather than using just vague slides. At present, there are many treatment methods for GBM patients, including resection, radiation, chemotherapy, immunotherapy, and antiangiogenic therapy, such as bevacizumab (BVZ) treatment (trade name Avastin or BEV). Despite such treatment options, to date, early detection and accurate classification of tumors is still the most effective method to achieve the best efficacy for patients.

MicroRNAs (miRNAs) are small noncoding RNAs that are approximately 22 nucleotides long, but they can bind to messenger RNAs (mRNAs) and mainly play an inhibitory role in gene expression in a posttranscriptional manner. Previous studies have shown that changes in miRNA expression in tumor tissues and body fluids are different from those in normal tissues or in different GBM subtypes. These changes involve various aspects of GBM, including tumor initiation, aggressiveness, responses to drug treatments, and patient survival rates. Profiling and studying these miRNA expression differences can help to further classify GBM. For example, based on miRNA and mRNA expression, five clinically and genetically distinct glioblastoma subclasses were classified, including oligoneural, radial glial, neural, neuromesenchymal, and astrocytic precursor glioblastoma. However, all these glioma subtypes classified with multiple miRNAs are difficult to accurately detect by traditional biomarkers and thresholds. In addition, there are fewer classifications related to drug responses, especially for bevacizumab (BVZ) treatment.

BVZ treatments target vascular endothelial growth factor A (VEGF), which can significantly reduce the early-stage tumor diameters of some GBM patients based on imaging studies. Anti-angiogenic treatment can significantly reduce the early stage of contrast enhancement during imaging studies and is therefore considered to have a high radiological response rate. However, BVZ treatment did not prolong the overall survival of GBM patients with these radiological responses. Also, BVZ alone or in combination with treatment had a significant effect on the transcriptional changes and tumor size decreases in its responders, with approximately 30-33% of all GBM patients; but changes in non-responders were small. Despite this, many physicians still believe that certain patients seem to benefit significantly from bevacizumab treatment, so more research should have been conducted to better identify this patient subgroup. Researchers also want to know what happens to these patients after receiving VEGF-targeting BVZ treatment, and what can be done for them in the near future.

For early detection and accurate classification of cancers, several machine learning algorithms have been applied to microarray datasets, including support vector machines (SVMs), random forest (RF), and neural network (NN). However, previous studies have shown that among popular techniques for multicategory classification of gene expression profiling datasets, SVMs have a dominant role, significantly outperforming all other methods, especially for small datasets and binary classification. SVMs were first introduced by Vapnik. Recently, it has shown effectiveness in many patterns' recognition problems, such as cancer histopathology image analysis and recognition, and they usually provide better classification performances than many other classification techniques. To identify GBM patients from normal patients, Teplyuk et al. reported using an SVM method based on miRNA expression levels in CSF to separate them. To date, there have been no successful reports using this method in combination with biomarkers to classify and detect cancer subtypes. To achieve better results using SVM methods in GBM classification, prediction, and detection, it is also very important to collect a certain number of datasets and select a suitable kernel function for SVMs. The heterogeneity of GBM complicates the classification of their responses to different treatments, especially considering races, different mutants and gene expression, and the clinical techniques used to obtain experimental datasets.

To address the aforementioned problems and needs, the present invention provides a unique solution in the form of new methods of detection and diagnosis and classification of GBM BVZ-subtypes using multiple biomarkers and artificial intelligence (AI) technology. Based on these findings, the present invention provides methods and means to prescreen GBM BVZ-responsive patients so as to avoid the aging-related side effects associated with unnecessary and even harmful treatment of such patients with BVZ. Importantly, the methods and products of the present invention provide detection, diagnosis, and classification methods of GBM BVZ-responsive subtypes using the multiple biomarkers and AI technology as disclosed herein that have a much higher accuracy rate than achievable using traditional methods with such biomarkers.

### SUMMARY OF THE INVENTION

The following listing of embodiments is a nonlimiting statement of various aspects of the invention. Other aspects and variations will be evident in light of the entire disclosure.

An aspect of the present invention relates to a panel of a group miRNAs used as biomarkers for the classification and detection of glioblastoma multiforme (GBM) bevacizumab (BVZ)-responsive subtypes for future clinical use. Further, the present invention provides endogenous control and standard curves for the selected miRNAs of the said panel. The present invention also provides a group of miRNAs, which are highly-relevant in terms of their differential expression to the GBM BVZ subtypes. The present invention additionally provides an experimental method for obtaining the expression of the miRNA panel in the GBM tissues, along with a formula and a calculation method to convert the obtained expression of the miRNA panel into data for use by an algorithm, where a set of machine learning algorithms that the present invention also provides lead to the detection of the GBM BVZ subtypes based on the differential expression of the miRNA panel in the GBM tissues. Moreover, the present invention provides some clinical characteristics of the GBM BVZ subtypes, including differential expression (DE) of miRNAs and mRNAs, survival times, vascular endothelial growth factor A (VEGF) methylation, and related functional pathways that result in precise treatment of GBM patients.

An aspect of the present invention provides a method for classification, clinical detection, and diagnosis of bevacizumab (BVZ)-responsive and non-responsive glioblastoma multiforme (GBM) subtypes referred to collectively as BVZ GBM subtypes by using machine learning algorithm, particularly, support vector machines (SVM) based methods in combination with miRNA biomarkers for classification, clinical detection, and diagnosis of GBM BVZ-responsive subtypes based on the expression of a miRNA panel of biomarkers obtained from existing clinical studies and transcription profiling. Moreover, based on the aforementioned precise investigation of the present invention, it further provides insights and results that relate to understanding of functional pathways related to VEGF and angiogenesis as they correlate with the patients belonging to the GBM BVZ-non-responsive subtype as compared with the patients belonging to the GBM BVZ-responsive subtype in terms of differences in survival rates and differences in differentially expressed (DE) mRNAs and different functional pathways leading to a new classification of regulatory angiogenesis mechanisms in GBM patients.

Further, another aspect of the present invention provides a method for classification and detection of GBM BVZ subtypes that includes the step of isolation of total RNA from GBM tissues, the evaluation of the quality and quantity of the obtained RNA, the reverse transcription (RT) of the obtained RNAs, and the real-time PCR amplification of miRNAs in the panel and the control, followed by the step of using the formula as disclosed herein, the standard curves, and the obtained miRNA expression to obtain experimental data for the machine learning algorithm, followed by the step involving machine learning algorithm as disclosed herein in combination with the obtained miRNA data from molecular experiment, to classify and detect the BVZ-responsive or BVZ-nonresponsive subtypes of GBM.

Additionally, an aspect of the present invention relates to combining of the miRNA biomarkers and machine learning classifier as disclosed herein to classify and detect BVZ GBM subtypes for clinical use, where one such clinical use is for prescreening and pre-detection of the BVZ-non-responsive GBM subtype of the GBM patients, where using BVZ as determined by the methods as disclosed in the present invention will cause adverse side effects, which are aging-related side effects that include aging, wound complications including dehiscence, CSF leaking, and infections after BVZ treatment in such patients when compared with before BVZ treatment and thus propagate the idea and method to avoid said side effects by prescreening and classifying such GBM patients as the BVZ-non-responsive GBM subtype to prevent them from receiving non-beneficial and unnecessary BVZ treatment.

Further, the methods provided by the present invention verify a new GBM subtype classification, after using various bioinformatics methods, differential expression (DE) of miRNAs and mRNAs, and their related clinical characteristics, pathways, and functions between the subtypes. Also, the present invention relates to several machine learning algorithms that were constructed and compared based on the miRNA expression z-scores and clinical datasets, and the SVM classifier as was selected for the present invention. To further use the classifier of the present invention, the panel of miRNAs was optimized, and the classifier was modified. After certain modification and validation, the present invention as disclosed has been further used for GBM clinical sample classification, detection, and further clinical and basic research. The present invention shows that subtypes of GBM BVZ response can be successfully classified and detected, so the treatment effects and related mechanisms could be reassessed individually for these different subtypes. Further, the present disclosure herein is useful to further explore possible mechanisms, effects, and side effects of BVZ treatment in GBM patients based on bioinformatics analysis.

The present invention thus provides biomarkers based on miRNA differential expression and methods to obtain experimental data from GBM tissues, convert the said experimental data to be used in the algorithm as disclosed herein to classify and detect the BVZ subtypes of GBM. The present invention is advantageous in the fact that instead of traditional diagnostic biomarkers and thresholds, the present invention provides miRNA biomarkers and machine learning algorithms that can be advantageously used to classify and detect the BVZ subtypes of GBM, where traditional diagnostics may not work.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The accompanying drawings are included to provide a further understanding of the invention, and are incorporated in and constitute a part of the present invention and, together with the description, serve to explain the principle of the invention.

In the drawings,

FIG. 1 depicts the experimental procedures. (A) The defined and classified BVZ subtypes of GBM. (B) This step includes several parts of the analyses. (C) The construction and comparison of classifiers based on the miRNA expression z-scores and clinical datasets. (D)

The optimization of a panel of miRNAs, including miR-21, miR-10b, and miR-197, and the modification and cross-validation of the SVM classifier based on clinical datasets. (E) The validation of the classifier using other datasets. In this figure, black lines and arrows indicate the workflow, and red lines and arrows indicate that the results obtained call back and support the previous work or definition.

FIG. 2 depicts an embodiment of the present invention providing the GBM BVZ subtypes and hierarchical clustering heatmaps of miRNAs and mRNAs. (A) The upregulated expression z-scores of miR-21 and miR-10b (UU) define the GBM BVZ subtype, which is present in ˜30% of all GBM patients. (B) The table shows all four subgroups of GBM, their labels, and percentages. (C) Hierarchical clustering heatmap of miRNAs in GBM BVZ and CON subtypes. The DE 14 miRNAs and their p-values are listed on the right of the map, p=<0.0001 vs the control, n=409. (D) Hierarchical clustering heatmap of mRNAs in these two GBM subgroups. Those seven mRNA IDs and their p-values are listed on the right of the map, p=<0.01 vs the control, n=409. Rulers of miRNA and mRNA expression are above the pictures, where −3 to 3 correspond to green to red. The yellow lines separate the GBM BVZ and its control subgroups, and the left sides of the lines are the GBM BVZ subtypes. If a p-value is less than 1.0E-10, the software will give zero as the p-value.

FIG. 3 depicts an embodiment of the present invention providing the distinct clinical characteristics of GBM BVZ subtypes. (A) Kaplan—Meier survival curves for the GBM BVZ subtype and the control (p=0.014, Wilcoxon-Mann). (B) Kaplan-Meier survival curves for GBM BVZ patients in each subclass (w or w/o MGMT methylation) of the entire subtype are shown (p>0.05). (C) Kaplan-Meier survival curves for GBM CON patients in each subclass are shown (p=0.043, Wilcoxon-Mann). Black or dashed lines represent the survival of patients with or without MGMT methylation, respectively. (D) Comparisons of the gene methylation of VEGF, VEGFB, and VEGFC between GBM BVZ subtypes are shown. There was only one significant difference between GBM BVZ and CON subtypes in VEGF methylation (p=0.005, Wilcoxon-Mann).

FIG. 4 depicts an embodiment of the present invention providing the constructing and comparing some classifiers for GBM BVZ subtypes based on miRNA z-scores of profiling. (A) For the z-score data points of the two subgroups, the horizon and vertical axes represent miR-21 and miR-10b. The blue or red dots are the GBM BVZ subgroup or the control subgroup. (B) The min. objective vs number of function evaluations during the construction of the SVM classifier. (C) The SVM model classifies GBM BVZ subtypes. All dots are the same as above, and the circles are the support vectors used. The curve is the edge of the two subgroups, and the accuracy is 100%, where the label ‘SV’ is Support Vector. (D) The RF algorithm classifies GBM BVZ subtypes. The dots are in the same labels. If a blue or a red dot is in the yellow or cyan region, the prediction is correct; otherwise, it is wrong.

FIG. 5 depicts an embodiment of the present invention and provides the relative miRNA expression in GBM tissues and cancer cell lines. In this study, the relative expression levels of four miRNAs, miR-21, miR-10b, miR-197, and miR-16, were used to detect GBM BVZ subtypes. The relative expression of miRNAs is expressed as the relative copy numbers of U6. In this picture, 10303_, 9746_, and 10099_ are GBM brain tissue samples, and U87 and mb are a GBM cell line and an MB cell line, respectively.

FIG. 6 depicts an embodiment of the present invention and provides the construction of SVM and RF classifiers for GBM BVZ subtypes based on clinical data. (A) The SVM model classified GBM BVZ subtypes. The blue or red dots represent the training data for the BVZ or CON subtypes. The circles are support vectors used. The curve is the edge of two groups, and the accuracy is 90.2%. (B) The RF model classified GBM BVZ subtypes with 82.9% accuracy. The blue or red dots represent the same thing as above. If a blue dot is in the yellow area, the prediction of the GBM BVZ subtype is correct; whereas if a red dot is in the cyan region, the prediction of the GBM CON subtype is correct. Otherwise, the prediction is wrong. (S1)

FIG. 7 depicts an embodiment of the present invention and provides the receive operating curve (ROC) and the confusion matrix for the SVM classifier cross-validation. (A) After 5-fold cross validation for the SVM classifier, the ROC was obtained, in which the red dot represented the average accuracy of 85.4%, sensitivity of 77.2%, and specificity of 88.8%. (B) The confusion matrix is shown for this cross-validation of the SVM classifier. (S2)

FIG. 8 depicts an embodiment of the present invention and provides the miR-197 expression z-scores in TCGA and GSE25631 datasets used. In TCGA dataset used, the miR-197 z-score is negative in the BVZ subtype and positive in the control subtype of GBM. In contrast, the miR-197 z-score is positive in the BVZ subtype and negative in the control subtype of GBM in the GSE25631 GBM dataset. (S3)

FIG. 9 depicts an embodiment of the present invention and provides that aging and VEGF related network in GBM BVZ-nonresponsive patients. (A) Venn diagram showing the relationship of aging associated genes, VEGF-associated genes, and DE mRNAs before/after BVZ treatment in GBM BVZ-nonresponsive patients. (B) Network between VEGF, aging-associated and DE mRNAs before/after BVZ treatment in GBM BVZ-nonresponsive patients.

FIG. 10 depicts an embodiment of the present invention and provides that three biomarkers including miR-21, miR10b, and miR197 when used for detection of GBM BVZ-responsive subtypes when comparing these multiple biomarkers used in combination with traditional and conventional methods of diagnosis to the method of detection and diagnosis of the present invention. The graphical data shows that using the method of the present invention when the three biomarkers including miR-21, miR10b, and miR197, are used in combination with a machine learning algorithm of the present invention as disclosed in an embodiment of the present invention, an appreciable accuracy rate of 95%, as shown by a red circle in this figure is achieved which is much greater than achieved by any of the traditional methods shown with different symbols as identified in the enclosed figure.

### DETAILED DESCRIPTION OF THE INVENTION

Detailed embodiments of the present invention are disclosed herein. However, it is to be understood that the disclosed embodiments are merely exemplary of the present invention, which may be embodied in various systems. Therefore, specific details disclosed herein are not to be interpreted as limiting, but rather as basis for teaching one skilled in the art to variously practice the present invention.

All illustrations of the drawings are for the purpose of describing selected versions of the present invention and are not intended to limit the scope of the present invention.

The present invention may be understood more readily by reference to the following detailed description of the invention taken in connection with the accompanying drawing figures, which forms a part of this disclosure. It is to be understood that this invention is not limited to the specific devices, medicines, systems, conditions or parameters described and/or shown herein and that the terminology used herein is for the example only, and is not intended to be limiting of the claimed invention. Also, as used in the specification including the appended claims, the singular forms ‘a’, ‘an’, and ‘the’ include the plural, and references to a particular numerical value includes at least that particular value unless the content clearly directs otherwise. Ranges may be expressed herein as from ‘about’ or ‘approximately’ another particular value. When such a range is expressed, it is another embodiment. Also, it will be understood that unless otherwise indicated, dimensions and material characteristics stated herein are by way of example rather than limitation, and are for better understanding of sample embodiment of suitable utility, and variations outside of the stated values may also be within the scope of the invention depending upon the particular application.

The terms “comprising,” “having,” “including,” and “containing” are to be construed as open-ended terms (i.e., meaning “including, but not limited to,” and permit the presence of one or more features or components) unless otherwise noted. It should be understood that while various embodiments in the specification are presented using “comprising” language, under various circumstances, a related embodiment may also be described using “consisting of” or “consisting essentially of language.

As such, the terms “a” (or “an”), “one or more,” and “at least one” can be used interchangeably herein. Furthermore, “and/or” where used herein is to be taken as specific disclosure of each of the two specified features or components with or without the other. Further, unless specifically stated or obvious from context, as used herein, the term “or” is understood to be inclusive. Also, unless specifically stated or obvious from context, as used herein, the terms “a”, “an”, and “the” are understood to be singular or plural.

As used herein, the term BVZ GBM subtypes or GBM BVZ subtypes refer to bevacizumab (BVZ)-responsive and non-responsive glioblastoma multiforme (GBM) subtypes, where BVZ GBM subtypes or GBM BVZ subtypes are used interchangeably and refer to the same thing.

In any of the ranges described herein, the endpoints of the range are included in the range. However, the description also contemplates the same ranges in which the lower and/or the higher endpoint is excluded. Additional features and variations of the invention will be apparent to those skilled in tire art from the entirety of this application, including the drawing and detailed description, and all such features are intended as aspects of the invention. Likewise, features of the invention described herein can be re-combined into additional embodiments that also are intended as aspects of the invention, irrespective of whether the combination of features is specifically mentioned above as an aspect or embodiment of the invention. Also, only such limitations which are described herein as critical to the invention should be viewed as such; variations of the invention lacking limitations which have not been described herein as critical are intended as aspects of the invention.

Unless defined otherwise, all technical and scientific terms used herein have the same meaning as commonly understood by one of ordinary skill in the art to which this disclosure is related.

Units, prefixes, and symbols are denoted in their Systems International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or aspects of the disclosure, which can be had by reference to the specification as a whole. The entire document is intended to be viewed as a unified disclosure, and it should be understood that all combinations of features described herein are contemplated. All references cited herein are hereby incorporated by reference in their entireties.

Embodiments will now be described in details with reference to the accompanying drawings. To avoid unnecessarily obscuring in the present disclosure, well-known features may not be described, or substantially the same elements may not be redundantly described, for example. This is for ease of understanding. The drawings and the following description are provided to enable those skilled in the art to fully understand the present disclosure and are in no way intended to limit the scope of the present disclosure as set forth in the appended claims.

As discussed hereinabove, there remains a problem and need for using a machine learning algorithm in combination with multiple biomarkers such as miRNA biomarkers to classify and detect cancer subtypes including for glioblastoma multiforme (GBM) and for use of such classification for further clinical use and application towards treatment options and what treatment to prevent by prescreening and classification of such cancer subtypes in cancer patients.

To address the abovementioned problems and needs in the area, the present invention provides a unique solution in the form of new methods of classification, clinical detection, diagnosis and prescreening of GBM cancer subtypes as it relates to bevacizumab (BVZ) treatment and responsiveness in the GBM patients using multiple biomarkers including miRNA and mRNA differential expression (DE) and z-scores in combination with machine learning algorithms.

The present invention provides methods for detection, classification, and prediction of GBM BVZ-responsive subtypes based on existing miRNA and mRNA profiling from the total RNA obtained from GBM tissues. The present invention discloses that compared with other GBM patients, patients with the GBM BVZ subtype had a significantly shorter survival time. After using multiple bioinformatics methods on these subtypes, differential expression (DE) miRNAs and mRNAs were obtained, and their related functions and clinical characteristics were analyzed. Finally, the SVM classifier and the miRNA panel of miR-21, miR-10b, and miR-197 were provided for future clinical use, and they were examined and validated with several datasets. The present invention provides methods and biomarkers based on miRNA that can be used to classify and detect GBM clinical samples, and it may propose precise diagnosis and treatment ideas for GBM.

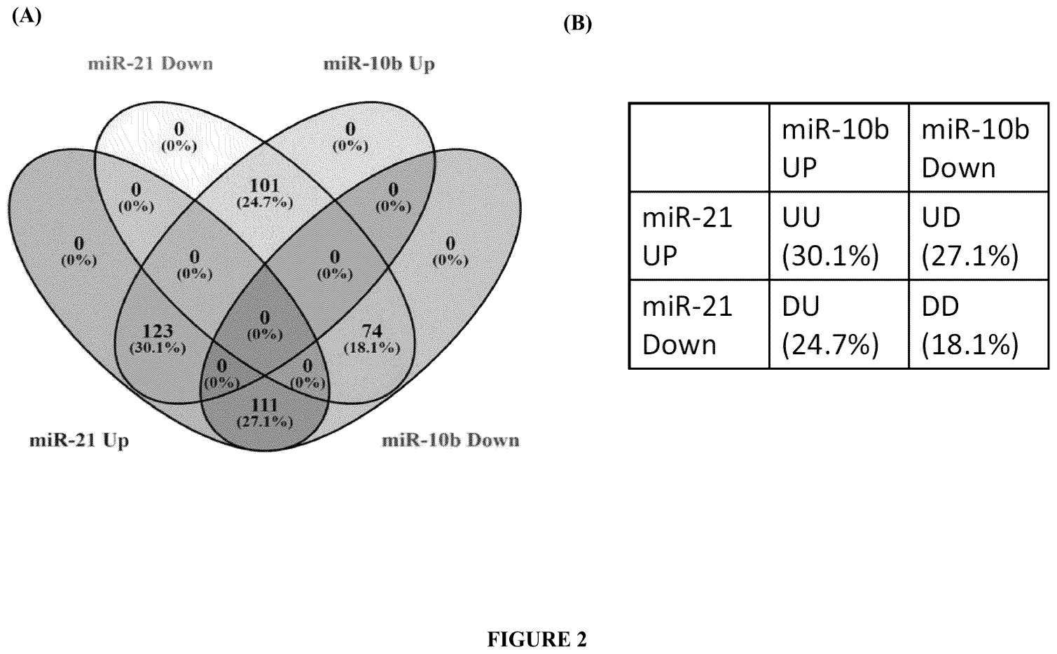

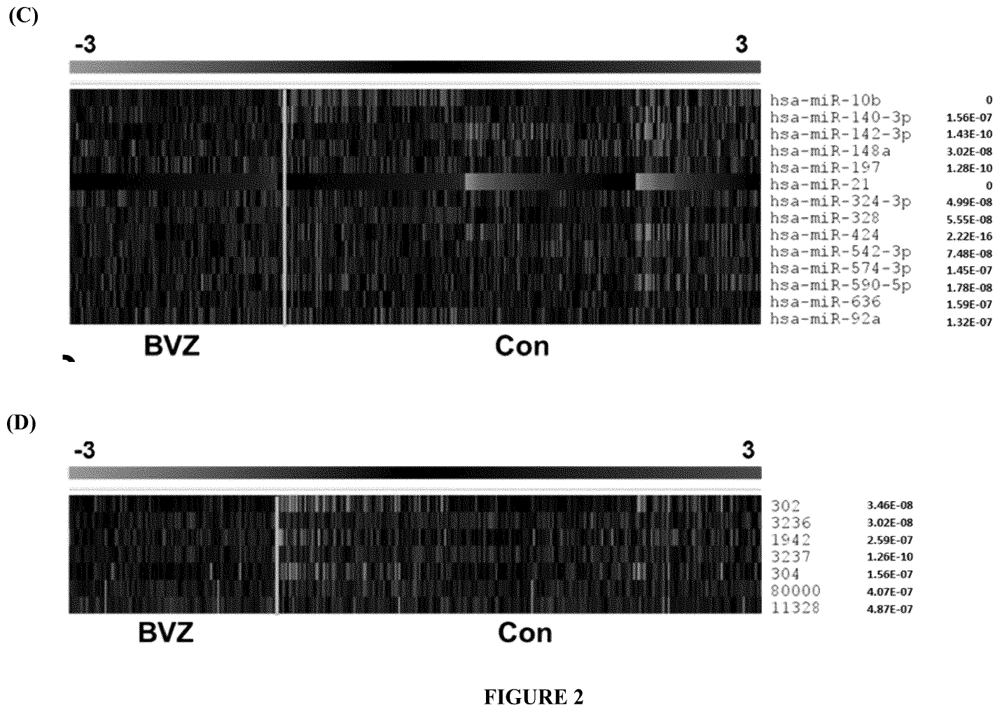

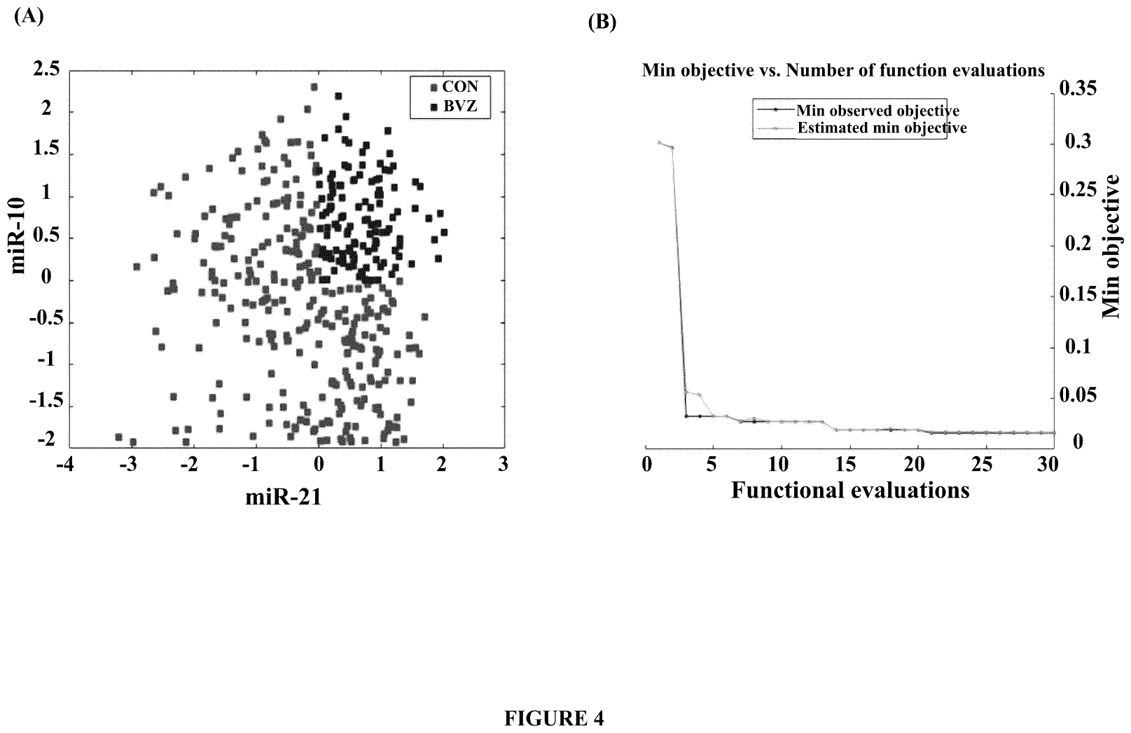

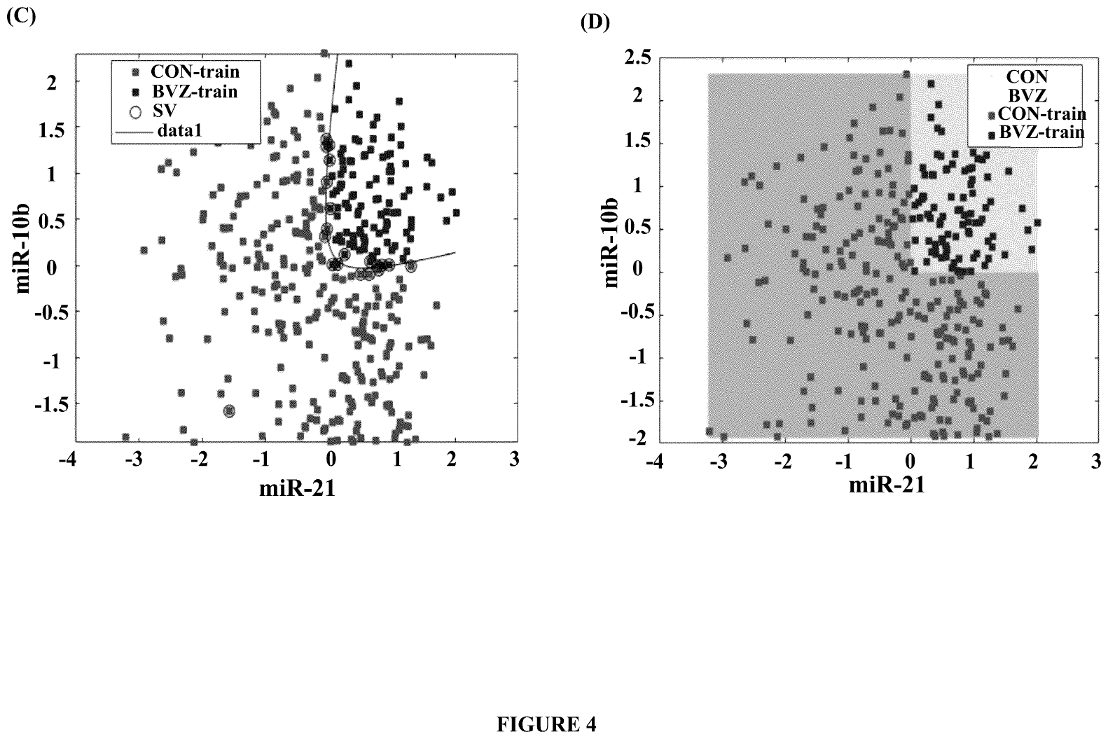

The present invention discloses that by combining machine learning methods with the miRNA panel, GBM BVZ subtypes could be classified and predicted by existing miRNA profiling datasets, and they may also be detected and predicted from GBM clinical data. Previously, several GBM subclasses were classified by multiple miRNA and mRNA expression profiling, but without artificial intelligence techniques, it is difficult to detect the classified GBM subclasses because multiple miRNAs cannot use some simple cutoff values of multiple biomarkers to separate different subclasses. As shown in FIG. 2B, there are four different subgroups for the expression of two miRNAs, and the actual edge between these two BVZ subtypes is a curve, not straight lines, as shown in FIG. 4C.

The present invention addresses the problems associated with precise diagnosis and treatment which are far more complicated than traditional diagnosis and treatment as understood by carrying out multiple validations for the present disclosure. The present invention relates to understanding of the problems that may affect the detection accuracy including experimental techniques, patient ethnicity, data scales and ranges and provides that the classifier must be modified and trained with the same dataset before use. The pretraining dataset used cannot be too small as disclosed by the present invention. For example, when using a dataset with 37 GBM samples (GSE19870) obtained by different microarray platforms, the present invention failed to obtain a stable high-accuracy classifier.

In addition, since high levels of miR-10b and miR-21 were observed in the serum of some glioblastoma patients before treatment and during BVZ treatment compared with healthy controls and other patients, this GBM BVZ subtype classification can be used not only to design therapeutic plans, but also to monitor treatment effects. Importantly, high expression levels of miR-10b and miR-21 were observed in the BVZ subtype of GBM.

According to the five subclasses classified by multiple miRNAs¹², patients with high expression of miR-21 and miR-10b were in different neural precursors or subgroups: astrocytic or oligoneural glioblastoma. These two glioblastoma subclasses have very different clinical and genetic distinctions. In addition, the combination of the panel of miR-21, miR-10b, and miR-197 with the SVM classifier for classification and detection of GBM BVZ subtypes has not been previously reported. Therefore, the present invention provides that this BVZ subtype is a new subtype of GBM. In addition, the present invention discloses that since the survival time of patients with the GBM BVZ subtype was significantly shorter than that of other patients, people need to consider this when evaluating the therapeutic effects of BVZ.

An embodiment of the present invention provides that 14 DE miRNAs were obtained between GBM BVZ subtypes, of which 7 have never been reported. Previously, miR-148a and miR-92a have been reported by our previous GBM study using the relative regression method. In another study, miR-92 was also reported. A further embodiment of the present invention thus relates to the presence of miR-21 and miR-10b among the miRNAs, where they function as a feedback support for the results obtained. It has been previously reported that as a tumor suppressor, downregulated miR-197 targeted GAB2 (Grb2-associated binding protein), releasing the inhibited proliferation in tumor cells. Further, in high-grade human gliomas, such as GBM, miR-197 was targeted and downregulated by downregulated FUS1 (TUSC2, tumor suppressor candidate 2), and downregulated miR-197 acted as a tumor suppressor to increase tumor cell proliferation.

An aspect of the present invention provides that the expression level of miR-542-3p negatively correlated with the invasion of glioblastoma cells by targeting AKT145, and different expression levels of miR-542 were present between GBM BVZ subtypes, as shown in FIG. 2C, which foes beyond what was known and reported by Cai et al. that miR-542-3p was downregulated in glioblastoma cell lines. Further, in contrast to Pang et al. that found that downregulation of miR-590-3p in GBM tissues and cell lines resulted in increased migration and invasion by targeting ZEB½ (zinc finger E-box ½), the present invention discloses that miR-590 expression was reversed between the BVZ subtype and the control of GBM. An additional aspect of the present invention provides that the unreported miRNAs and the reported miRNAs with unreported inverse expression patterns indicated very special miRNA expression patterns that are associated with these two GBM BVZ subtypes. This may be because other researchers used normal tissues as controls, whereas the present invention used other GBM patients as controls rather than GBM patients with the BVZ subtype. Thus, the present invention provides that these miRNAs are specifically associated with these GBM BVZ subtypes. For example, as shown in FIG. 2C, the present invention discloses that miR-197 expression was downregulated in the GBM BVZ subtype, but it was upregulated in its control subgroup, and so it was selected as one of the variants for the SVM classifier after comparing all other combinations by the sequencesf program. The present invention provides miR-16 as the endogenous control because miR-16 expression increased in GBM patients compared with normal controls and was maintained at the same expression level in GBM BVZ subtypes.

An aspect of the present invention provides that the different functional profiles based on the 14 DE miRNAs so obtained, in addition relates to common functions of miRNAs, wherein some related functions are associated with VEGF. For general functions, RNA binding and gene silencing are routine functions of miRNAs, as shown in Table 1, hereinbelow.

TABLE 1
Functional characteristics based on the differntial expressed
14 miRNAs. The table shows all significant regulatory pathways
between GBM BVZ subtypes based on the obtained 14 DE miRNAs by
using the g:Profier tool and setting the threshold at p = 0.01.

| Source | Term Name | −LOG10(p) |
|---|---|---|
| GO:MF | RNA binding involved in posttranscriptional | 7.044533686 |
| | gene silencing | |
| GO:MF | mRNA binding involved in posttranscriptional | 7.044533686 |
| | gene silencing | |
| GO:MF | mRNA binding | 5.379949536 |
| GO:MF | mRNA 3′-UTR binding | 4.999431533 |
| GO:MF | RNA binding | 2.636641753 |
| GO:BP | gene silencing by miRNA | 6.866746351 |
| GO:BP | post-transcriptional gene silencing by RNA | 6.816330049 |
| GO:BP | posttranscriptional gene silencing | 6.791483042 |
| GO:BP | gene silencing by RNA | 6.647180023 |
| GO:BP | gene silencing | 6.187240965 |
| GO:BP | posttranscriptional regulation of gene expression | 4.741993024 |
| GO:BP | negative regulation of gene expression | 3.540780231 |
| GO:BP | regulation of blood vessel endothelial cell | 3.319663972 |
| | proliferation in sprouting angiogenesis | |
| GO:BP | aortic smooth muscle cell differentiation | 3.252517619 |
| GO:BP | regulation of aortic smooth muscle cell | 3.252517619 |
| | differentiation | |
| GO:BP | blood vessel endothelial cell proliferation | 3.155460339 |
| | involved in sprouting angiogenesis | |
| GO:BP | miRNA mediated inhibition of translation | 2.419265504 |
| GO:BP | negative regulation of translation, ncRNA- | 2.419265504 |
| | mediated | |
| GO:BP | regulation of translation, ncRNA-mediated | 2.406850992 |
| GO:CC | extracellular space | 2.136945023 |

An aspect of the present invention discloses that after analyzing the GO functional terms of all 14 DE miRNAs, several GO terms were clearly associated with VEGF, including regulation of blood vessel endothelial cell proliferation in sprouting angiogenesis (GO:1903587), blood vessel endothelial cell proliferation involved in sprouting angiogenesis (GO:0002043), and aortic smooth muscle cell differentiation (GO:0035887). These functions are very specific to VEGF, which is the design principle of the GBM BVZ subtypes and the feedback support for the results. Furthermore, if only 8 of the 14 miRNAs with increased expression were searched, more functions were associated with VEGF than previous functions, including regulation of endothelial cell proliferation (GO:0001936), endothelial cell proliferation (GO:0001935), and sprouting angiogenesis (GO:0002040). The reasons for the increased VEGF-related functions are unclear, suggesting that some functions may be hedged by negatively expressed miRNAs. However, the present invention provides that if only negatively expressed miRNAs were analyzed, they had fewer relevant functions, suggesting that increased expression of miRNAs has dominant functions as inhibitors of mRNA expression, but the converse may not be the general case.

An aspect of the present invention provides a method to analyze the mRNA and miRNA expression patterns associated with bevacizumab-responsive glioblastoma subtypes, and provides the existence of new GBM subtypes with clinical characteristics, DE miRNAs and mRNAs, and related specific functions.

Another aspect of the present invention provides a method that combine the SVM classifier and the panel of miRNAs to not only classify but also detect GBM BVZ subtypes for future clinical use. In addition, the present invention provides that miR-21, miR-10b, and miR-197 can be used as potential GBM BVZ subtype biomarkers or monitors to help make therapeutic decisions and monitor treatment.

### Experimental Procedure

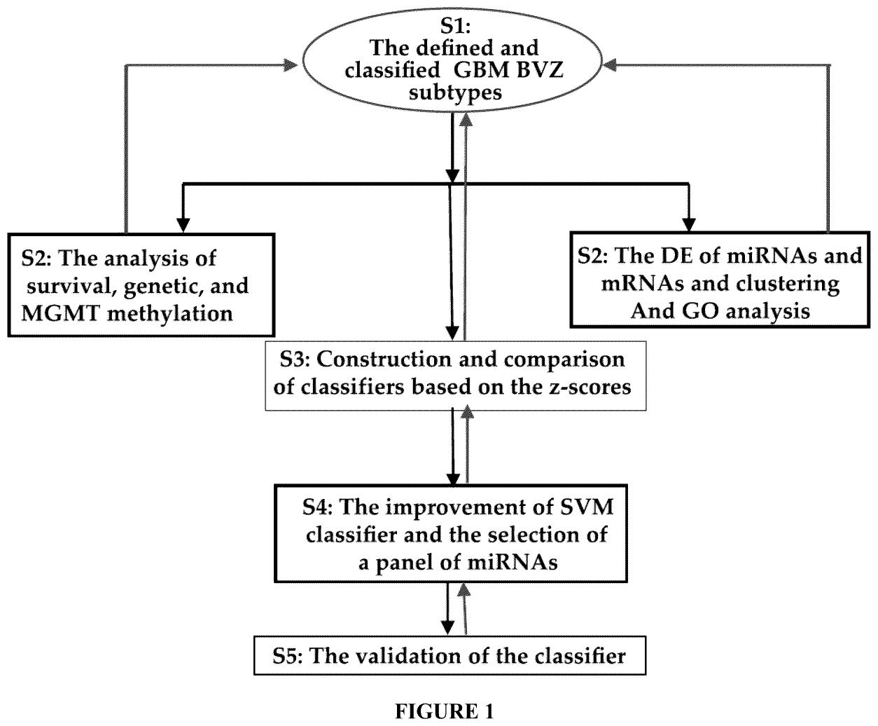

The experimental procedure included the following steps, as shown in FIG. 1.

The first step was to define and classify the GBM BVZ-responsive subtype (abbreviated as the GBM BVZ subtype). According to the following criteria, all patients were divided into the GBM BVZ subtype and its control subgroup (BVZ nonresponsive) by using the Venn diagram method. In the 2nd step, the GBM BVZ subtype was assessed, demonstrated, and compared to its control subtype through multiple bioinformatic and clinical analyses, including Kaplan-Meier survival curves, miRNA and mRNA clustering, clinical characteristics, and genetic and functional analyses. In the 3rd step, based on the expression z-scores of miR-21 and miR-10b in GBM BVZ subtypes, several machine learning algorithms, such as SVM, RF, and NN, were constructed and compared, and the SV classifier was selected, in which the radial basisi function (RBF) was used as the kernel function. The 4th step was to prepare the SVM classifier for clinical use, find the best combination of miRNAs, perform cross-validation, and modify the classifier. Finally, the SVM classifier was examined and validated by using several datasets. In FIG. 1, black lines represent the workflow, and red lines represent the feedback support of the results.

### Experimental Datasets

In the Cancer Genome Atlas (TCGA) pilot study, mRNA and miRNA expression profiles of GBM were generated using Affymetrix U133A, Affymetrix Exon 1.0 ST, custom Agilent 244 K, and Agilent miRNA array platforms. Several mRNA expression profiles were integrated into a single estimate of relative gene expression for each gene in each sample¹³. From TCGA portal (http://cancergenome.nih.gov/dataportal/), the expression z-scores of miRNA and mRNA and miRNA expression profiling were downloaded, especially including data_expression_merged_median_Zscore and data_expression_miRNA, and additional datasets were also downloaded, including clinicopathological annotations and methylation for glioblastoma patients.

### Software and Statistical Methods

In the present invention , the software and online tools used included MATLAB (R2021b and R2022a, MathWorks Inc., Natick, MA), R-project (version 4.0.4, www.r-project.org), MeV (version 4.9.0, MEV, LLC., Walnut Creek, CA, http://www.tm4.org/mev.html), the Venny diagram¹³, Gene Ontology (http://geneontology.org/), g:Profier (ELIXIR, Tartu, Estonia, https://biit.cs.ut.ee/gprofiler/gost), and SigmaPlot14 (Systat Software, San Jose, CA). The present invention used statistical methods that included Student's t-test, the standard Bonferroni adjusted t-test, and the Wilcoxon-Mann-Whitney test if the data distribution was not the standard distribution.

### Definition and Classification of GBM BVZ Subtypes

The present invention defined the BVZ-responsive GBM subtype (abbreviated as the GBM BVZ subtype) as patients with high expression of miR-21 and miR-10b in their tumors. The GBM BVZ subtype was defined because high levels of miR-10b and miR-21 were observed in the serum, cerebrospinal fluid (CSF), and tumor tissues of some glioblastoma patients compared with normal control patients. Most importantly, the expression levels of miR-10b and miR-21 were negatively correlated and significantly associated with decreased tumor diameters in the BVZ treated group, but not in the temozolomide (TMZ)-treated group.

High levels of miR-10b or miR-21 were defined as z-scores greater than zero, and low levels of miR-10b or miR-21 were defined as z-scores below or zero. Patients were selected and subdivided into four groups according to the expression z-scores of miR-10b and miR-21 using the R program. Then, using the Venn diagram²², the patient IDs of the GBM BVZ subtype with high expression of both miR-21 and miR-10b were obtained, while the patient IDs of other groups were also obtained, including the group with both miR-21 and miR-10b downregulated group (DD), miR-21 upregulated and miR-10b downregulated group (UD), and miR-21 downregulated and miR-10b upregulated group (DU). All three subgroups were combined as either the GBM BVZ nonresponsive subtype (abbreviated as the CON subtype) or the GBM control subtype.

### Hierarchical Clustering of miRNAs and mRNAs of GBM BVZ Subtypes

Based on patient IDs of GBM BVZ subtypes (BVZ and CON), the z-score matrices of miRNAs and mRNAs were obtained using the R program, and patients' GBM BVZ subtypes were also labelled. Then, clustering and heatmaps of miRNAs and mRNAs were obtained by using MeV program^{24,25}. Since two subgroups were given before clustering, this hierarchical clustering belongs to semi-unsupervised learning.

### Cell Culture and GBM Tissues

Four cancer cell lines were cultured in this study. All cell lines were purchased from the American Type Culture Collection (ATCC). All experiments followed the Biological Use Authorization (BUA) of VA Medical Center at San Francisco, California. The human U87 GBM cell line was cultured in 10% FBS in DMEM (Invitrogen, USA). Medulloblastoma (MB) cells (CHLA-01-MED) were cultured in DMEM: F12 medium (ATCC 30-2006) with 20 ng/mL human recombinant EGF, 20 ng/mL human recombinant basic FGF, and B-27. The PC3 prostate cancer cell line was cultured in 10% FBS in F-12 (Invitrogen, USA), and the DU145 prostate cancer cell line was cultured in 10% FBS in DMEM (Invitrogen, USA)¹⁵. Three fresh human glioblastoma multiform specimens were collected from patients, including GBM10302 (age 49, female), GBM 9746 (age 70, male), and GBM10099 (age 55, male). Informed consent was obtained from all the participants included in the study. All tissue samples were obtained during the initial resection, and none of the patients had received prior chemotherapy or radiation therapy. The samples were immediately cut into pieces estimated at 30-40 mg per piece and stored in a −80° C. freezer until use.

### Ethics Approval and Consent to Participate

Informed consent was obtained from all three human samples. The ethics approval number of the Local of Ethics Committee of University of California is 10-01318, which is used to de-identify human biospecimens. These studies were in accordance with the ethical standards of the institutional research committee and with the 1964 Helsinki Declaration and its later amendments.

### Quantitative Real-Time PCR

Total RNA from all experimental cells and tissues was isolated by using a mirVana™ miRNA Isolation Kit (Thermo Fisher, USA), and the RNA quality and quantity were evaluated using a NanoDrop 2000 (Thermo Scientific, Waltham, MA). Reverse transcription (RT) of the RNAs was performed by using a first-strand cDNA synthesis system (Invitrogen, USA), according to the manufacturer's protocol.

Real-time PCR amplification was performed using a real-time PCR kit (TaKaRa, USA) following the manufacturer's protocol. For all experiments, PC3 and DU145 cells were used as negative and technical controls to keep the machine settings, reagents and techniques at the same level for each experiment. The test samples were subjected to 40 cycles of PCR amplification. The experiments were performed using the QuantStudio7 and 7900HT Fast Real-time PCR system (Applied Biosystems). All experiments were repeated in duplicate for all samples and all miRNAs.

### Data Availability

The raw datasets analyzed during the current study are available in The Cancer Genome Atlas (TCGA) repository [http://cancergenome.nih.govidataportal/].

An embodiment of the present invention provides a method for classification, clinical detection, and diagnosis of bevacizumab (BVZ)-responsive and non-responsive glioblastoma multiforme (GBM) subtypes referred to collectively as BVZ GBM subtypes based on a panel of a group of micro RNAs (miRNAs) to be used as biomarkers in combination with computer implemented machine learning algorithms for said classification clinical detection, and diagnosis of the BVZ GBM subtypes, the method comprising the steps of:

(i) obtaining the expression z-scores of miRNAs and mRNAs and miRNA expression profiling data for GBM patients including data_expression_merged_median_Zscore and data_expression_miRNA downloaded from the Cancer Genome Atlas (TCGA) pilot study datasets, and downloading additional datasets, including clinicopathological annotations and methylation data for the GBM patients;

(ii) defining, classifying, and selecting the GBM patients in terms of BVZ responsiveness into the BVZ GBM subtypes which are identified and classified as a BVZ-responsive GBM subtype and a BVZ-non-responsive GBM subtype also referred to as control for GBM patient selection;

(iii) assessing, demonstrating, and comparing the obtained BVZ-responsive GBM subtype to the BVZ-non-responsive GBM subtype by analyzing the clinicopathological annotations and methylation data for the GBM patients including survival time, heredity and mutations, and methylation, and the expression z-scores of miRNAs and mRNAs and miRNA expression profiling data for differential expression (DE) of miRNAs and mRNAs, clustering and GO analysis thereof to obtain analysis results applicable for further GBM patient selection and classification into the BVZ GBM subtypes;

(iv) performing data statistical analysis on the DE miRNAs of the step (iii) by performing a t-test on the obtained z-score matrix and comparing the BVZ GBM subtypes to determine which miRNAs differed most between the BVZ-responsive GBM subtype and the BVZ-non-responsive GBM subtype to obtain analyzed DE miRNAs;

(v) ranking the analyzed DE miRNAs of the step (iv) to obtain highly relevant miRNAs for the BVZ GBM subtypes;

(vi) performing hierarchical clustering of DE miRNAs between the BVZ GBM subtypes and then visualizing the clustering as a heatmap using MeV software to visualize the highly relevant miRNAs for each of the BVZ GBM subtypes, including, the BVZ-responsive GBM subtype and the BVZ-non-responsive GBM subtype;

(vii) performing data statistical analysis on the DE mRNAs of the step (iii) by performing a t-test on the obtained z-score matrix and comparing the BVZ GBM subtypes to determine which mRNAs and consequently their coded genes differed most between the BVZ-responsive GBM subtype and the BVZ-non-responsive GBM subtype to identify significant genes for the BVZ GBM subtypes;

(viii) performing hierarchical clustering of the significant genes between the BVZ GBM subtypes and then visualizing the clustering as a heatmap using MeV software to visualize the significant genes in terms of DE mRNAs for each of the BVZ GBM subtypes, including, the BVZ-responsive GBM subtype and the BVZ-non-responsive GBM subtype;

(ix) constructing three machine learning algorithms and comparing the three machine learning algorithms;

(x) preparing, implementing, modifying, and optimizing supervised machine learning classifiers for each of the three machine learning algorithms of the step (ix) based on the defined and classified BVZ GBM subtypes and the matrices obtained based on the steps (ii) to (viii) above based on z-scores for DE of miRNAs, finding the best combination of miRNAs, performing cross-validation, and modifying said classifiers to train and test a first set of clinical datasets;

(xi) constructing further of the supervised machine learning classifiers of the step (x) for each of the three machine learning algorithms of the step (ix) for clinical use based on current clinical techniques without the use of z-scores to classify, detect, and diagnose the BVZ GBM subtypes;

(xii) comparing the constructed supervised machine learning classifiers as constructed in the step (xi) for accuracy rate resulting in selection of the constructed SVM classifier from the step (xi) for further clinical use;

(xiii) optimizing and evaluating further of the constructed SVM classifier from the step (xi) to obtain a new and improved machine learning process of the SVM classifier for best accuracy rate and results in identification of a panel of a group of miRNAs to be used as biomarkers for the classification and clinical detection of the BVZ GBM subtypes obtained as the best combination with a step-by-step optimization and evaluation process for the highly relevant miRNAs of the step (v);

(xiv) performing stratified k-fold cross-validation for the SVM classifier to prevent overfitting;

(xv) obtaining the confusion matrix, sensitivity, specificity, and mean accuracy on which the receiver operating curve (ROC) of the SVM classifier was generated, and the said SVM classifier was used for further validation;

(xvi) validating the SVM classifier of the step (xv) by using a second set of clinical datasets,

wherein the defining, classifying, and selecting the GBM patients of the step (ii) in terms of BVZ responsiveness into four subgroups based on the expression levels or z-scores of miR-21 and miR-10b as obtained by using the R program and a Venn diagram method,

wherein among the four subgroups, the patient IDs of the GBM patients with high expression of both miR-21 and miR-10b are identified as a first subgroup, and defined and classified as the BVZ-responsive GBM subtype, while the patient IDs of other three subgroups of the GBM patients, which include a second subgroup with both miR-21 and miR-10b downregulated referred to as DD group, a third subgroup with miR-21 upregulated and miR-10b downregulated referred to as UD group, and a fourth subgroup with miR-21 downregulated and miR-10b upregulated referred to as DU group, are identified, combined, defined, and classified as the BVZ-non-responsive GBM subtype,

wherein the BVZ-responsive GBM subtype are the GBM patients who are highly responsive to BVZ treatment, while the BVZ-non-responsive GBM subtype did not highly respond to BVZ treatment,

wherein the comparing of the three machine learning algorithms in the step (ix) is done using the same dataset and based on the expression z-scores of miR-21 and miR-10b, represented by a batch of miRNA expression datasets, and

wherein the analysis the steps (iii) to (viii) further established the expression changes in levels of miR-21 and miR-10b as important for classification of the GBM patients into distinct subgroups.

Another embodiment of the present invention provides a system for classification, clinical detection, and diagnosis of bevacizumab (BVZ)-responsive and non-responsive glioblastoma multiforme (GBM) subtypes referred to collectively as BVZ GBM subtypes based on a panel of a group of micro RNAs (miRNAs) to be used as biomarkers in combination with computer implemented machine learning algorithms for said classification clinical detection, and diagnosis of the BVZ GBM subtypes.

Another embodiment of the present invention provides a panel of a group of micro RNAs (miRNAs) to be used as biomarkers for classification, clinical detection, and diagnosis of bevacizumab (BVZ)-responsive and non-responsive glioblastoma multiforme (GBM) subtypes referred to collectively as BVZ GBM subtypes.

Another embodiment of the present invention provides a panel of a group of micro RNAs (miRNAs) for use as biomarkers in combination with computer implemented machine learning algorithms for classification, clinical detection, and diagnosis of bevacizumab (BVZ)-responsive and non-responsive glioblastoma multiforme (GBM) subtypes referred to collectively as BVZ GBM subtypes.

Another embodiment of the present invention provides the method for classification, clinical detection, and diagnosis of the BVZ GBM subtypes as disclosed herein, wherein the defining, classifying, and selecting the GBM patients in the step (ii) of the method for classification, clinical detection, and diagnosis of the BVZ GBM subtypes as disclosed herein, is based on the high expression levels of miR-21, and miR-10b defined as z-scores greater than zero, while low levels of miR-10b or miR-21 are defined as z-scores below or zero, and wherein the high expression levels of miR-21, and miR-10b, were negatively correlated and significantly associated with decreased tumor diameters in the BVZ treated group, but not in a temozolomide (TMZ)-treated group based on the data from the datasets of the step (i) of the method for classification, clinical detection, and diagnosis of the BVZ GBM subtypes as disclosed herein.

Another embodiment of the present invention provides the method for classification, clinical detection, and diagnosis of the BVZ GBM subtypes as disclosed herein, wherein the defining, classifying, and selecting the GBM patients in the step (ii) of the method for classification, clinical detection, and diagnosis of the BVZ GBM subtypes as disclosed herein is based in terms of BVZ responsiveness in the serum, cerebrospinal fluid (CSF), and tumor tissues of the GBM patients as compared with normal control patients based on the data from the datasets of the step (i) of the method for classification, clinical detection, and diagnosis of the BVZ GBM subtypes as disclosed herein.

Another embodiment of the present invention provides the method for classification, clinical detection, and diagnosis of the BVZ GBM subtypes as disclosed herein, wherein the ranking of the analyzed DE miRNA in the step (v) of the method for classification, clinical detection, and diagnosis of the BVZ GBM subtypes as disclosed herein is done by a p-value with a cutoff at 0.0001 and standard Bonferroni correction for each subtype to obtain the highly relevant miRNAs for the BVZ GBM subtypes that pass the threshold, and said highly relevant miRNAs include miR-10b, miR-140-3p, miR-142-3p, miR-148a, miR-197, miR-21, miR-324-3p, miR-328, miR-424, miR-542-3p, miR-574-3p, miR-590-5p, miR-636, and miR-92a.

Another embodiment of the present invention provides the method for classification, clinical detection, and diagnosis of the BVZ GBM subtypes as disclosed herein, wherein the identifying significant genes in the step (vii) of the method for classification, clinical detection, and diagnosis of the BVZ GBM subtypes as disclosed herein, is performed using Student's t-test at a p-value with a cutoff at 0.01 and with false discovery correction, standard Bonferroni correction referred to as FDC analysis to obtain the significant genes for the BVZ GBM subtypes that pass the threshold between these two subgroups as gene signatures of GBM BVZ subtypes, and said significant genes include annexin A2 (ANXA2), homeobox D10 (HOXD10), ephrin Al (EFNA1), homeobox D11 (HOXD11), annexin A2 pseudogene 2 (ANXA2P2), GREB1 like retinoic acid receptor coactivator (GREB1L), and FKBP prolyl isomerase 9 (FKBP9).

Another embodiment of the present invention provides the method for classification, clinical detection, and diagnosis of the BVZ GBM subtypes as disclosed herein, wherein the assessing, demonstrating, and comparing in the step (iii) of the method for classification, clinical detection, and diagnosis of the BVZ GBM subtypes as disclosed herein further established differences between the two BVZ GBM subtypes including the BVZ-responsive GBM subtype and the BVZ-non-responsive GBM subtype in terms of differences in survival time, wherein said BVZ-responsive GBM subtype as obtained and classified according to the high expression of miR-21 and miR-10b in GBM patients is identified as a new BVZ subtype of GBM, absent in any of the subclasses based on other miRNA consensus clustering, and wherein the mean survival time of the GBM patients classified as the BVZ-responsive GBM subtype is significantly shorter than that of the BVZ-non-responsive GBM subtype indicating that the BVZ-responsive GBM subtype is more aggressive than the BVZ-non-responsive GBM subtype.

Another embodiment of the present invention provides the method for classification, clinical detection, and diagnosis of the BVZ GBM subtypes as disclosed herein, wherein the three machine learning algorithms in the step (ix) of the method for classification, clinical detection, and diagnosis of the BVZ GBM subtypes as disclosed herein include support vector machine (SVM), random forest (RF), and neural network (NN), and wherein the respective methods are fitcsvm for SVM, fitctree for RF, and fitcnet for NN.

Another embodiment of the present invention provides the method for classification, clinical detection, and diagnosis of the BVZ GBM subtypes as disclosed herein, wherein the supervised machine learning classifiers of step (x) of the method for classification, clinical detection, and diagnosis of the BVZ GBM subtypes as disclosed herein for SVM is done using the fitcsvm program and radial basis function (RBF) as its kernel function, wherein using a random number generator, the dataset was divided into an 80% training set and a 20% testing set to train and test each of the datasets in the first set of clinical datasets, wherein the hyperparameters are automatically optimized by using the fitcsvm, in which the cross-validation loss was minimized.

Another embodiment of the present invention provides the method for classification, clinical detection, and diagnosis of the BVZ GBM subtypes as disclosed herein, wherein the further of the supervised machine learning classifiers of the step (x) of the method for classification, clinical detection, and diagnosis of the BVZ GBM subtypes as disclosed herein for each of the three machine learning algorithms of the step (ix) for clinical use based on current clinical techniques without the use of z-scores involves selecting miR-16 as the endogenous control, and the use of the following formula 1 for the analysis of expression data for any one of the miRNAs identified as miR-i from a microarray expression dataset calculated to the log2-transformed ratios, and performed as follows:

Ri=log2[(miR-i)/(miR-16)] Formula 1

where, Ri is the transformed ratio of control miR-16, and miR-i is the expression values of any one of the miRNAs being considered in this analysis for clinical use without the use of z-scores.

Another embodiment of the present invention provides the method for classification, clinical detection, and diagnosis of the BVZ GBM subtypes as disclosed herein, wherein the optimizing and evaluating further of the constructed SVM classifier is done by the use of the sequentialfs function to add and try more variants for better prediction to reach the new and improved machine learning process of the SVM classifier for best accuracy rate obtained herein, wherein the optimizing and evaluating further of feature subsets based on the highly relevant miRNAs of the step (v) of the method for classification, clinical detection, and diagnosis of the BVZ GBM subtypes as disclosed herein is done using formula 2 as follows:

sequentialfs(fun, X, y) Formula 2

where, said formula 2 selects a subset of features from the data matrix X and sequentially compares features until the best candidate is found to best predict the data in y.

Another embodiment of the present invention provides the method for classification, clinical detection, and diagnosis of the BVZ GBM subtypes as disclosed herein, wherein the step-by-step optimization and evaluation of each possibility and each combination for each of the highly relevant miRNAs of the step (v) of the method for classification, clinical detection, and diagnosis of the BVZ GBM subtypes as disclosed herein leads to the best combination that can be obtained from this process which is identified as the panel of a group of miRNAs to be used as biomarkers for the classification and clinical detection of the BVZ GBM subtypes.

Another embodiment of the present invention provides the method for classification, clinical detection, and diagnosis of the BVZ GBM subtypes as disclosed herein, wherein the panel of a group of miRNAs to be used as biomarkers for the classification and clinical detection of the BVZ GBM subtypes includes miR-21, miR-10b, and miR-197.

Another embodiment of the present invention provides the method for classification, clinical detection, and diagnosis of the BVZ GBM subtypes as disclosed herein, wherein wherein the stratified k-fold cross-validation is performed using crossvalind and fitcsvm programs.

Another embodiment of the present invention provides the method for classification, clinical detection, and diagnosis of the BVZ GBM subtypes as disclosed herein, wherein the second set of clinical datasets for validating the SVM classifier in the step (xvi) of the method for classification, clinical detection, and diagnosis of the BVZ GBM subtypes as disclosed herein comprise different miRNA microarray datasets.

Another embodiment of the present invention provides the method for classification, clinical detection, and diagnosis of the BVZ GBM subtypes as disclosed herein, wherein the second set of clinical datasets for validating the SVM classifier in the step (xvi) of the method for classification, clinical detection, and diagnosis of the BVZ GBM subtypes as disclosed herein comprise real-time quantitative polymerase chain reaction (qPCR) data obtained from total RNA isolated from tissues of GBM patients in molecular experiments

Another embodiment of the present invention provides the method for classification, clinical detection, and diagnosis of the BVZ GBM subtypes as disclosed herein, wherein the classification, clinical detection, and diagnosis of the BVZ GBM subtypes based combining of multiple miRNA biomarkers in the form of the panel of the group of miRNAs to be used as biomarkers with computer implemented machine learning algorithms achieves an accuracy rate of at least 95%, which is suitable for successful use in clinical detection and application, whereas traditional methods using one or more of said miRNA biomarkers of the panel including miR-21, miR-10b, and miR-197, in any combination thereof, with the respective thresholds achieve an accuracy rate that is too low and unsuitable for use in clinical detection and application for classification, detection, diagnosis, and prediction of the BVZ GBM subtypes

An embodiment of the present invention provides a diagnostic method for detection of bevacizumab (BVZ)-responsive and non-responsive glioblastoma multiforme (GBM) subtypes referred to collectively as BVZ GBM subtypes comprising combining a panel of a group of miRNAs to be used as biomarkers to classify, detect, diagnose, and predict BVZ-responsiveness in GBM tissues obtained from GBM patients with machine learning algorithms, the method comprising the steps of:

(a) obtaining tissues from subjects;

(b) isolating total RNA from the tissues in the step (a);

(c) evaluating the quality and quantity of the isolated total RNA in the step (b);

(d) performing reverse transcription (RT) on the isolated total RNA after its evaluation in the step (c);

(e) performing real-time quantitative polymerase chain reaction (qPCR) amplification of miRNAs after RT of the step (d);

(f) obtaining copy numbers to quantify amplification of selected miRNAs with respect to endogenous control RNAs calculated in terms of Ct values and average standard curves to obtain copy numbers from the qPCR amplification performed in the step (e);

(f) using a normalized equation as follows: En=Copy number (target)/Copy number (reference), wherein, the target is any one of the selected miRNAs from the step (f) and reference is an endogenous control, which is U6 RNA, to obtain the relative expression levels target miRNAs;

(g) using the formula 1 and calculation process as follows:

Ri=log2[(miR-i)/(miR-16)] Formula 1

to convert the obtained relative expression levels of the target miRNAs referred to as miR-i in the formula 1 and represents any one of the experimental miRNAs of a panel of a group of miRNAs to be used as biomarkers to classify, detect, classify, and predict BVZ-responsiveness in GBM tissues obtained from GBM patients in comparison to the endogenous control miRNA of formula 1, miR-16 into data, where, Ri is the log2-transformed ratio of control miR-16;

(h) combining multiple miRNA biomarkers with machine learning algorithms by using the data obtained in the step (g) as input dataset for a support vector machine (SVM) classifier as obtained in the claim 1 for machine learning algorithms that leads to the classification, detection, diagnosis, and prediction of the BVZ GBM subtypes based on the differential expression (DE) pattern of the experimental miRNAs of the panel of the group of miRNAs to be used as biomarkers in the GBM tissues,

wherein subjects comprise patients with GBM, and malignant glioma,

wherein the tissues comprise serum, cerebrospinal fluid (CSF), and tumor tissues,

wherein the tissues can be fresh or frozen,

wherein the experimental miRNAs in the panel of the group of miRNAs to be used as biomarkers to classify, detect, diagnose, and predict BVZ-responsiveness in GBM tissues obtained from GBM patients comprise miR-21, miR-10b, and miR-197, and

wherein the combining multiple miRNA biomarkers with machine learning algorithms in the step (h) achieves an accuracy rate of at least 95%, which is suitable for successful use in clinical detection and application, whereas traditional methods using one or more said miRNA biomarkers of the panel including miR-21, miR-10b, and miR-197, in any combination thereof, with the respective thresholds achieve an accuracy rate that is too low and unsuitable for use in clinical detection and application for classification, detection, diagnosis, and prediction of the BVZ GBM subtypes.

An embodiment of the present invention provides a method for prescreening of patients with glioblastoma multiforme (GBM) referred to as GBM patients to prevent adverse aging-related side effects caused due to treatment and therapy with an anti-angiogenetic treatment, bevacizumab (BVZ) therapy for GBM patients that targets vascular endothelial growth factor A (VEGF) based on bioinformatics analysis, the method comprising the steps of:

(i′) obtaining mRNAs and mRNAs expression profiles for GBM patients downloaded from the Cancer Genome Atlas (TCGA) pilot study datasets, and downloading additional datasets, including clinicopathological annotations and methylation data for the GBM patients, and obtaining other datasets of GBM patients from gene expression omnibus (GEO) with sequencing datasets before and after BVZ treatment comprising expression data of mRNAs for the same patient before and after BVZ treatment;

(ii′) performing analysis using multiple bioinformatics and statistical methods including Venn diagram analysis, STRING (versionll) analysis, g: Profer (ELIXIR, Tartu, Estonia, https://biit.cs.ut.ee/gprofiler/gost) analysis, gene ontology (GO) and Kyoto encyclopedia of genes and genomes (KEGG) analysis, using software including MATLAB, R-project, and the statistical methods including Student's t-test and paired t-test on the datasets for the GBM patients of the step (i′);

(iii′) using the method of the claim 1 for classification of the datasets of the step (i′) into the BVZ GBM subtypes of the claim 1 classified as BVZ-responsive GBM subtype and BVZ-non-responsive GBM subtype;

(iv′) analyzing differential expression (DE) of mRNAs for differential gene expression patterns and BVZ-related networks before and after BVZ treatment as obtained from the analysis in the step (ii′) for the GBM patients classified as the BVZ-responsive GBM subtype and the BVZ-non-responsive GBM subtype based on the step (iii′) to obtain, assess, and analyze a BVZ treatment response;

(v′) performing paired t-tests before and after BVZ treatment on the two BVZ GBM subtypes after classifying and dividing the GBM patients into the BVZ-responsive GBM subtype and the BVZ-non-responsive GBM subtype in the step (iv′) to obtain significantly differentially expressed mRNAs and corresponding genes for each subtype;

(vi′) performing gene ontology (GO) and KEGG pathway analysis of the significantly differentially expressed mRNAs and corresponding genes as obtained in the step (v′) for the GBM patients classified as the BVZ-responsive GBM subtype and the BVZ-non-responsive GBM subtype to obtain gene expression patterns and functional pathways associated with BVZ treatment as obtained in analyzed data after the BVZ treatment;

(vii′) assessing, analyzing, and comparing the results of gene expression patterns and functional pathways obtained in the step (vi′) after the BVZ treatment for the GBM patients classified as the BVZ-responsive GBM subtype and the BVZ-non-responsive GBM subtype;

(viii′) identifying and assessing a relationship between aging-associated genes, VEGF-associated genes, and DE mRNAs before and after BVZ treatment in the BVZ-non-responsive subtype of the GBM patients based on the results of gene expression patterns and functional pathways of the step (vii′) to identify and assess the adverse aging-related side effects caused when BVZ is administered for treatment in the BVZ-non-responsive subtype of the GBM patients;

(ix′) prescreening and pre-detection of the GBM patients to be classified, detected, and diagnosed as the BVZ-non-responsive subtype based on a combination of biomarkers and machine learning algorithms of the claim 1 to prevent said adverse aging-related side effects of the step (viii′) from being caused due to BVZ treatment and therapy in prescreened and pre-detected BVZ-non-responsive subtype of the GBM patients and for careful selection of the GBM patients who are the BVZ-responsive subtype for initiation of BVZ treatment,

wherein the significantly differentially expressed mRNAs and corresponding genes in the step (v′) are obtained using a p-value with a cutoff at 0.05 for each subtype,

wherein the results of gene expression patterns and functional pathways associated with BVZ treatment as obtained in analyzed data after the BVZ treatment in the step (vi′) are obtained using a p-value with a cutoff at 0.01 for the BVZ-responsive GBM subtype,

wherein the results of gene expression patterns and functional pathways associated with BVZ treatment as obtained in analyzed data after the BVZ treatment in the step (vi′) lead to no functional pathways or specific gene expression patterns that crossed a threshold using a p-value with a cutoff at 0.05 for the BVZ-non-responsive GBM subtype,

wherein the results of gene expression patterns and functional pathways associated with BVZ treatment as obtained in analyzed data after the BVZ treatment in the step (vi′) for the BVZ-responsive GBM subtype indicate that after BVZ treatment, the gene expression patterns and related functional pathways in the BVZ-responsive GBM patients are specific and beneficial to the patients, and

wherein the results of gene expression patterns and functional pathways associated with BVZ treatment as obtained in analyzed data after the BVZ treatment in the step (vi′) for the BVZ-non-responsive GBM subtype indicate that after BVZ treatment, the gene expression patterns and related functional pathways in the BVZ-non-responsive GBM patients are not specific and not beneficial to the patients.

An embodiment of the present invention provides a system for prescreening of patients with glioblastoma multiforme (GBM) referred to as GBM patients to prevent adverse aging-related side effects caused due to treatment and therapy with an anti-angiogenetic treatment, bevacizumab (BVZ) therapy for GBM patients that targets vascular endothelial growth factor A (VEGF) based on bioinformatics analysis, comprising classification, clinical detection, and diagnosis of bevacizumab (BVZ)-responsive and non-responsive glioblastoma multiforme (GBM) subtypes referred to collectively as BVZ GBM subtypes based on a panel of a group of micro RNAs (miRNAs) to be used as biomarkers in combination with computer implemented machine learning algorithms for said classification clinical detection, and diagnosis of the BVZ GBM subtypes.

Another embodiment of the present invention provides the method for prescreening of GBM patients to prevent adverse aging-related side effects caused due to treatment and therapy with BVZ as disclosed herein, wherein the BVZ treatment response in the step (iv′) of the method for prescreening of GBM patients to prevent adverse aging-related side effects caused due to treatment and therapy with BVZ as disclosed herein is obtained, assessed, and analyzed according to the RANO criteria and confirmed on the subsequent follow-up MRI including complete or partial response (CR+PR).

Another embodiment of the present invention provides the method for prescreening of GBM patients to prevent adverse aging-related side effects caused due to treatment and therapy with BVZ as disclosed herein, wherein the adverse aging-related side effects include aging, wound complications including dehiscence, CSF leaking, and infections

Another embodiment of the present invention provides the method for prescreening of GBM patients to prevent adverse aging-related side effects caused due to treatment and therapy with BVZ as disclosed herein, wherein the DE mRNAs before and after BVZ treatment in the BVZ-non-responsive subtype of the GBM patients in the step (viii′) of the method for prescreening of GBM patients to prevent adverse aging-related side effects caused due to treatment and therapy with BVZ as disclosed herein showed significant expression changes and decrease in the levels of mRNAs and corresponding genes including Ephrin type A receptor 1 (EPHA1), endothelial cell specific molecular 1 (ESM1), and gremin 1 (GREM1) after BVZ treatment when compared to before BVZ treatment.

The invention will be further explained by the following Examples, which are intended to purely exemplary of the invention, and should not be considered as limiting the invention in any way.

### EXAMPLES

### Example 1

### GBM Sample Cohort

In this GBM sample cohort, there were 564 samples (544 untreated and 20 treated samples from 220 female and 344 male patients). In this example, 409 GBM samples containing both mRNA and miRNA profiles and z-scores for each sample from untreated patients were analyzed. All patients had been diagnosed with glioblastoma multiforme, some of which had more pathological information, including survival time, heredity and mutations, and methylation.

### Example 2

### Classification and Clustering of miRNAs and mRNAs for GBM BVZ Subtypes

Among all GBM patients, the percentage of patients with the GBM BVZ subtype is very important for follow-up studies. Based on the defined criteria and using the Venn diagram tool²², the distribution of all glioblastoma patients was grouped into four subgroups, as shown in FIGS. 2A and B. A total of 123 patients were classified as the GBM BVZ-responsive subtype, representing 30.1% of all GBM patients, who were highly responsive to BVZ treatment. Most importantly, a previous study showed that 33% of GBM patients responded to BVZ treatment compared to normal controls.

The other 286 patients, classified as the BVZ nonresponsive subtype of GBM, did not highly respond to BVZ treatment. According to the biological definition, the present example focused on these two GBM subtypes in the following studies. In addition, 286 control GBM patients were combined with three other subgroups, DD, UD, and DU, as shown in FIG. 2B.

Some DE miRNAs and mRNAs were found after the comparisons between GBM BVZ subtypes (BVZ and CON subtypes). After patient selection, DE miRNA analysis was performed by performing a t-test on the obtained z-score matrix to determine which miRNAs differed most between the subtypes. Following data statistical analysis, miRNAs were ranked by p-value with a cut-off at 0.0001 and standard Bonferroni correction for each subgroup. Specifically, 14 miRNAs between GBM BVZ subgroups passed the threshold as highly relevant miRNAs, including miR-10b, miR-140-3p, miR-142-3p, miR-148a, miR-197, miR-21, miR-324-3p, miR-328, miR-424, miR-542-3p, miR-574-3p, miR-590-5p, miR-636, and miR-92a. Hierarchical clustering and DE miRNAs between subgroups were then visualized as a heatmap using MeV software, as shown in FIG. 2C. In this heatmap, these highly relevant miRNAs and their p-values are listed on the right, the GBM BVZ subgroup is on the left of the yellow line, and the control subgroup is on the right.

For mRNA DE analysis, clusters between these GBM BVZ subgroups were examined. Based on the matrix of these GBM BVZ subtypes classified above, significant genes were picked using Student's t-test at p=0.01 and FDC (false discovery correction, standard Bonferroni correction). Specifically, seven genes were found to pass the statistical threshold between these two subgroups as gene signatures of GBM BVZ subtypes. These seven gene IDs are 302 (ANXA2), 3236 (HOXD10), 1942 (EFNA1), 3237 (HOXD11), 304 (ANXA2P2), 80000 (GREB1L), and 11328 (FKBP9). DE gene expression between these two subgroups was represented as a heatmap using MeV software, as shown in FIG. 2D. In this map, the significant gene IDs and their p-values are listed on the right side of the map. These DE expression levels of miRNAs and mRNAs may suggest that the expression changes of miR-21 and miR-10b in glioblastoma have an important role in classifying glioblastoma patients into distinct subgroups.

### Example 3

### Clinical Characteristics of GBM BVZ Subtypes

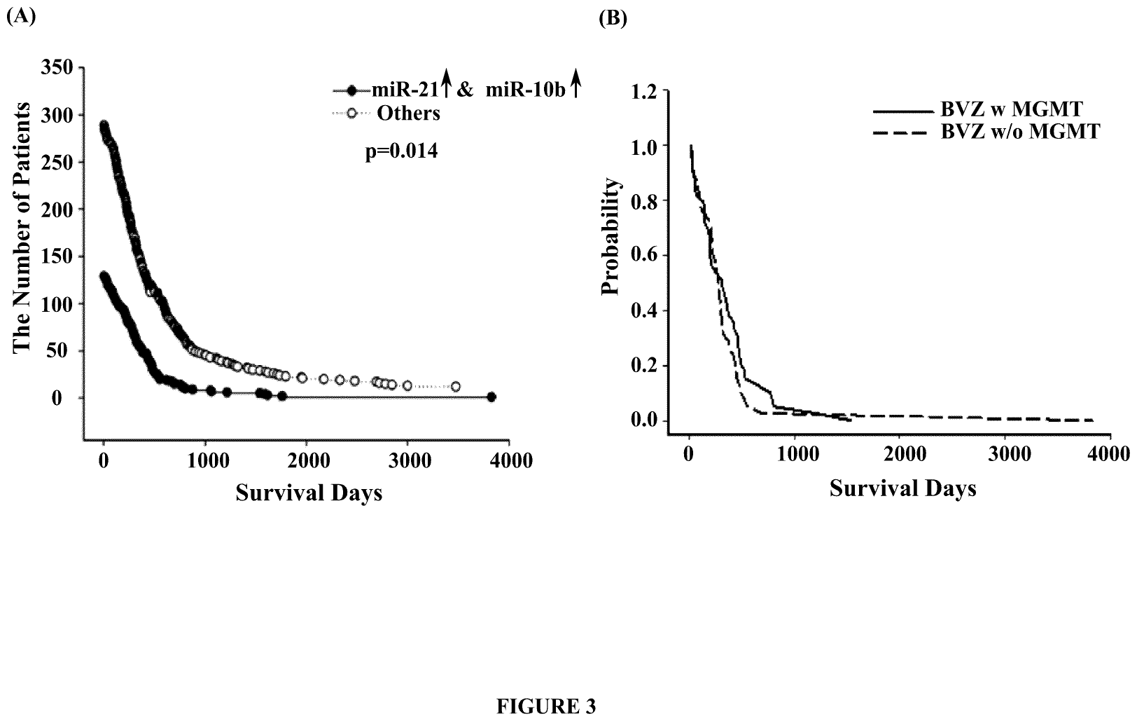

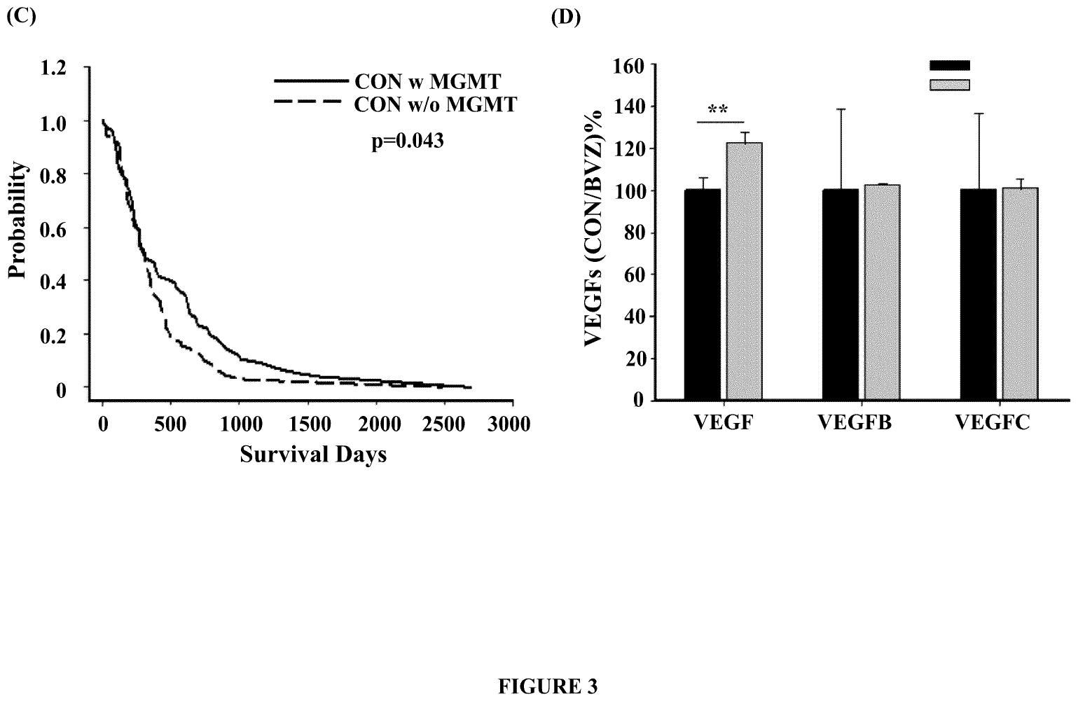

Differences in survival time between the two classified GBM BVZ subtypes suggested and supported the existence of a new BVZ subtype of GBM. GBM BVZ subtypes classified according to the high expression of miR-21 and miR-10b were absent in any of the subclasses based on miRNA consensus clustering; thus, the GBM BVZ-responsive subtype is a new subtype of GBM. Based on this classification of glioblastoma, patients' survival days were assessed and compared. Survival data were available for 121 patients in the GBM BVZ subgroup with a mean survival time of 295.9 days and for 277 patients in the BVZ control subgroup with a mean survival time of 355.1 days. Using these survival data and the Kaplan-Meier method, we compared the survival terms of these two subgroups, as shown in FIG. 3A. The present example found that patients with the GBM BVZ subtype had significantly shorter survival times than control patients (p=0.014, case=398). The average survival time between these two subgroups was shortened by approximately 60 days, indicating that the GBM BVZ subtype is more aggressive than the GBM control subtype.

In addition, the survival times of the other groups were also compared. Compared with the other three groups of DD, UD, and DU groups, there was only one significant difference (p=0.023) between the GBM BVZ (UU) and DD subgroups; that is, both miR-21 and miR-10b were downregulated. Previously, it was found that miR-21 was upregulated in many cancers and correlated with survival time in glioma patients, so all patients with miR-21 upregulation (UU+UD) were compared to all patients with downregulated miR-21 (DU+DD). There was no significant change, possibly because all patients in this experiment were GBM patients, excluding patients with low-grade gliomas. Furthermore, GBM patients with only upregulated miR-10b did not affect their survival time.

The experiment further compared the survival time of GBM patients associated with MGMT promoter methylation in these two subtypes (FIGS. 3B and C). It was reported that tumors carrying MGMT promoter methylation have a more favorable prognosis for GBM patients. However, these two GBM BVZ subtypes showed distinct survival patterns from MGMT promoter methylation. In the GBM control subtype but not the BVZ subtype, a significantly longer survival time association with MGMT promoter methylation was observed (p=0.043).

Further, the gene methylation of VEGF, VEGFB, and VEGFC was assessed since we studied the BVZ response in GBM patients. Overall, VEGF methylation was much lower than that of VEGFB and VEGFC. However, there was a significant decrease in VEGF methylation in the GBM BVZ subtype compared to the control subtype (p=0.005, case=284), and there were no significant differences in VEGFB and VEGFC, as shown in FIG. 3D. This may contribute to differences in survival and BVZ response between these two GBM subtypes.

### Example 4

### Functional Analysis Using DE miRNAs

The functional characteristics of those DE miRNAs between these GBM BVZ subtypes were examined. Functional profiles of global co-DE miRNAs (i.e., significantly up- and downregulated miRNAs across all GBM samples between these two GBM subtypes) were examined using Gene Ontology (GO) enrichment analysis. As expected, these 14 DE miRNAs were involved in mRNA binding and gene silencing (molecular function (MF)), regulation of cytokine production (biological process (BP)), regulation of blood vessel endothelial cell proliferation in sprouting angiogenesis (BP), and more. More details are shown in the Table 1 hereinabove. However, when dividing these 14 DE miRNAs into globally upregulated and downregulated groups, and the present invention found that these eight upregulated miRNAs are annotated in almost all functions that existed in the global analysis. Among the six downregulated miRNAs, one related to GO:1905001 had a different function than before, namely negative regulation of membrane repolarization during atrial cardiac muscle cell action potential (BP).

### Example 5

### Constructing Three Machine Learning Algorithms to Classify and Predict GBM BVZ Subtypes Based on miRNA Z-Scores

Based on the defined and classified GBM BVZ subtypes and the obtained matrices, this example implemented a supervised machine learning classifier, the support vector machine (SVM) to train and test the previously defined datasets. In this study, we used the fitcsvm program and radial basis function (RBF) as its kernel function. The expression z-scores of miR-21 and miR-10b and the labels of those two subgroups were used in this step, as shown in FIG. 4A. Using a random number generator, the dataset was divided into an 80% training set and a 20% testing set. Given a set of training examples, each labeled as belonging to one of two categories, the SVM training algorithm built a model that assigned new examples to one category or the other. To build the model, the hyperparameters were automatically optimized by using the fitcsvm, in which the cross-validation loss was minimized.

The optimization processes of the SVM model are shown in FIG. 4B, in which the number of function evaluations is the iteration number of the objective function, and the main objective is the minimum value of the objective function reached in that iteration. The built model reached 100% accuracy on the test set, as shown in FIG. 4C. In this figure, those circles represent the support vectors used, the dots represent the training data, and the curve completely separates the two subgroups.

To compare different machine learning algorithms, the RF classifier was constructed by using the fitctree function and the same dataset. The results are shown in FIG. 4D, and its accuracy also reached 100%. In this figure, the blue or red dots represent the BVZ or CON training data, and if a blue or red dot is within the yellow or cyan region, the prediction is correct; otherwise, the prediction is wrong. In addition, an NN classifier was constructed by using the fitcnet program, optimizing to contain 15 first-layer nodes and 10 s-layer nodes, which also achieved 100% accuracy on the z-score dataset.

However, all these perfect classifications were based on the expression z-scores of miR-21 and miR-10b, which were represented by a batch of miRNA expression datasets 13 and were difficult to use clinically.

### Example 6

### Constructing the SVM Classifier to Classify and Detect GBM BVZ Subtypes Based on Clinical Datasets

To diagnose these GBM BVZ subtypes clinically, we have to develop a detection method for the subtypes based on current clinical techniques without the use of z-scores. In a previous study, to classify GB BVZ subtypes, many datasets and controls were used. However, for the clinical tests so far, typically quantitative real-time PCR (qPCR) methods have been used to diagnose patient samples without control samples. The qPCR analysis for testing miRNA expression is based on the use of endogenous controls for standardization, reliability and reproducibility of results. For this purpose, we have to choose a control miRNA. After evaluating several candidates, miR-16 was selected for two reasons: first, the expression of miR-16 was relatively high in all studied samples, and second, its expression level was stable in GBM subgroups, and its SD was relatively low compared to the target miRNAs. After selecting miR-16 as the endogenous control, the microarray expression data selected in the above analysis were calculated to the log2-transformed ratios, which were performed as follows:

Ri=log2[(miR-i)/(miR-16)] Formula (1)

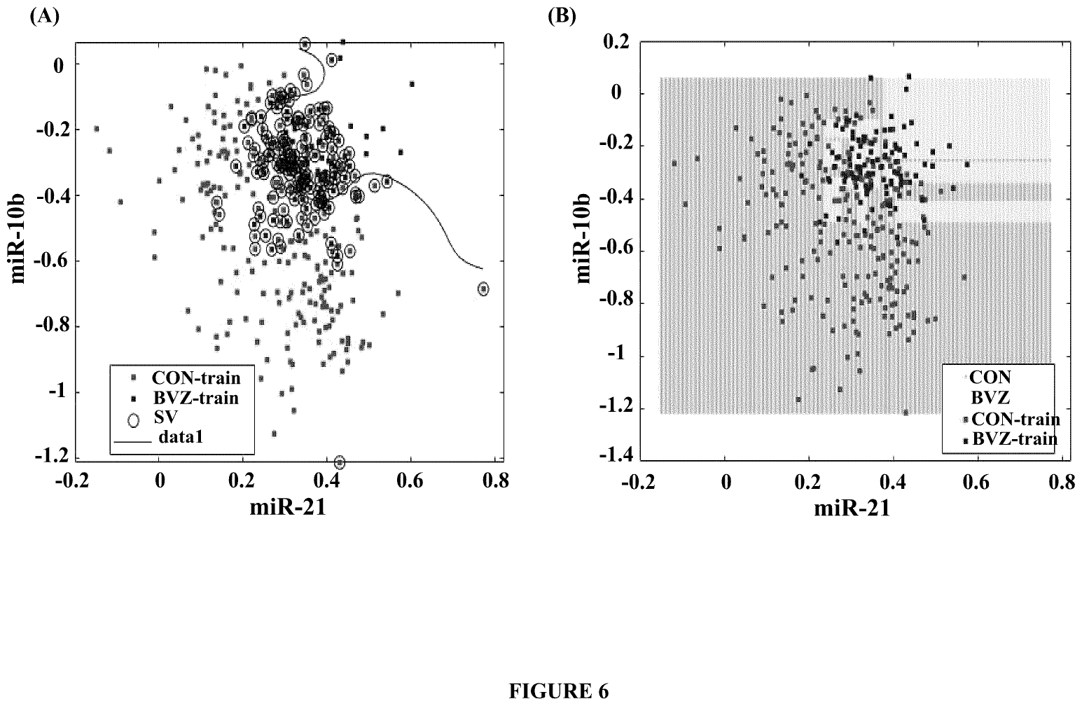

to this formula, the expression values of miR-21 and miR-10b were divided by miR-16, and then the log2 of the ratio was calculated. There were some overlaps in this dataset compared to the same dataset of z-scores. After using this data set, the same methods of SVM (fitcsvm), RF (fitctree) and NN (fitcnet) were used as above, so they were still supervised machine learning problems. The SVM classifier achieved 90.2% accuracy rate, while the RF classifier achieved an 82.9% accuracy rate, the results of which are shown in FIGS. 6A and B. Furthermore, the NN classifier achieved an accuracy of 83.9%. Therefore, the SVM classifier was selected in the following research. Although this SVM classifier can be used for prediction, better accuracy can be achieved by optimizing and adding some variants.

To better predict the subtypes, we would like to add and try more variants. For this purpose, the sequentialfs function was used, and the variants were automatically optimized and selected among the 14 miRNAs obtained above. The optimization of feature subsets is that sequentialfs(fun, X, y) selects a subset of features from the data matrix X and sequentially compares features until the best candidate is found to best predict the data in y.

In this example, the rows of the X matrix corresponded to patient observations; the columns of X corresponded to the 14 miRNAs obtained. In the first step, the sequentialfs selected each of the 14 miRNAs, kept calling the function and compared the accuracy until the best miR-21 was found. In the second step, the sequentialfs picked out each of the remaining 13 miRNAs to add miR-21 and consisted of each of the two miRNA panels, and then performed the same as before until the best two miRNA panel of miR-21 and miR-10b was found. Step-by-step evaluation of each possibility and each combination, including a panel of the 14 miRNAs, the best combination obtained from this process was the panel of miR-21, miR-10b, and miR-197. According to formula (1), the dataset for these three miRNA panel was calculated, and a new machine learning process of the SVM classifier was performed. This time, the prediction was indeed better than before, with the best accuracy rate reaching 95.1%. The overfitting problem of the SVM classifier was assessed by a cross-validation method before being used on other clinical datasets.

### Example 7

### K-Fold Cross Validation for the SVM Classifier

To prevent overfitting, stratified k-fold cross-validation for the SVM classifier was performed since the distribution of the two subtypes was not equal. In this process, the crossvalind and fitcsvm programs were used. After fivefold cross validation, the average accuracy reached 85.4% (±2.75, n=5), suggesting no overfitting problem. In fact, most people think that SVM classifiers with rbf as its kernel function have difficulty producing the overfitting problem.

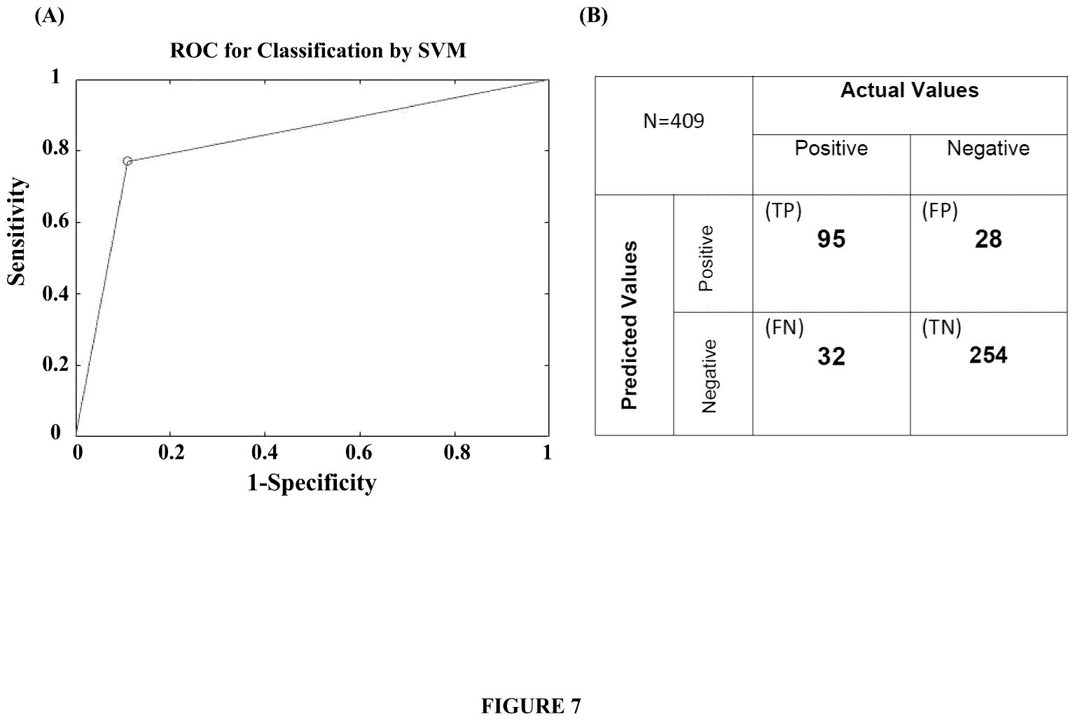

After the cross validation, the confusion matrix, sensitivity, specificity, and mean accuracy of 85.4% were obtained, on which the receiver operating curve (ROC) of the classifier was generated, as shown in FIG. 7A, and the confusion matrix is shown in FIG. 7B. This classifier was then used for validation.

### Example 8

### Validation of the SVM Classifier by Using Clinical Datasets

To use this classifier widely, we tested several kinds of datasets, including different miRNA microarrays and real-time qPCR data. First, we used the miRNA expression dataset of 20 treated patients from the same dataset used for classifier construction, since there were no significant changes in miRNA expression compared to untreated miR-21, miR-10b, miR-197, and miR-16. After using the classifier and the dataset directly, it was found that six patients belonged to the GBM BVZ subtype with a percentage of 30%, which is similar to the defined percentage we showed above. This validation seems to be very successful.

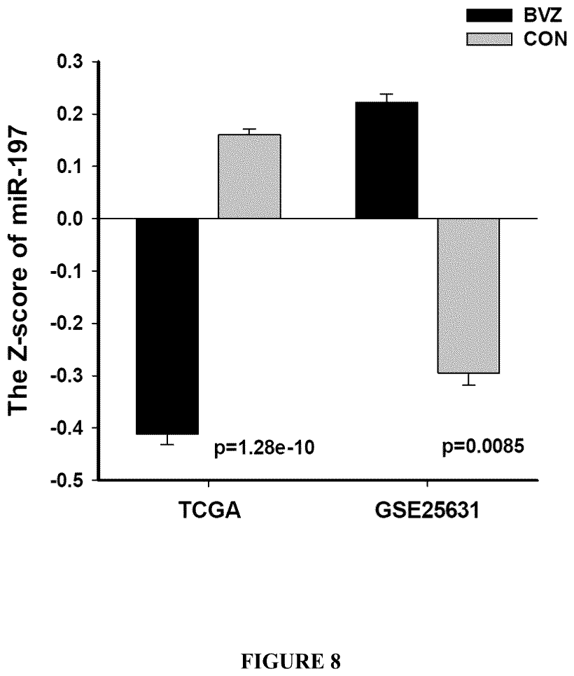

For the second validation, another miRNA expression profile (GSE25631) for malignant glioma tissues was used, in which GBM patients were Chinese, while the patients in the TCGA dataset used were mostly Caucasian patients. Although the microarray chips and detection methods were from the same company, the expression patterns of miRNAs were different. For example, the expression pattern of miR-197 was opposite to the microarray that was used above (as shown in FIG. 8), suggesting that miRNA expression may be different in different races. For this validation, we divided 81 GBM datasets (excluding one outlier) into 71 modification datasets and 10 validation datasets. The classifier was trained and modified using the dataset of 71 GBM samples until it reached 92.3% accuracy. After that, the classifier was used to detect the 10 remaining sample datasets, and the results were perfect for 3 BVZ patients and 7 control patients with GBM. If the experimental methods and patients' race are the same as the dataset used, this classifier can be directly used to detect GBM BVZ subtypes.

For the third validation, we performed a real-time qPCR experiment to obtain each Ct value of miR-21, miR-10b, miR-197, miR-16 and U6 from three GBM tissues, one GBM cell line, and one child brain tumor cell line.

The example found that the copy numbers of miRNAs can be used for this evaluation.

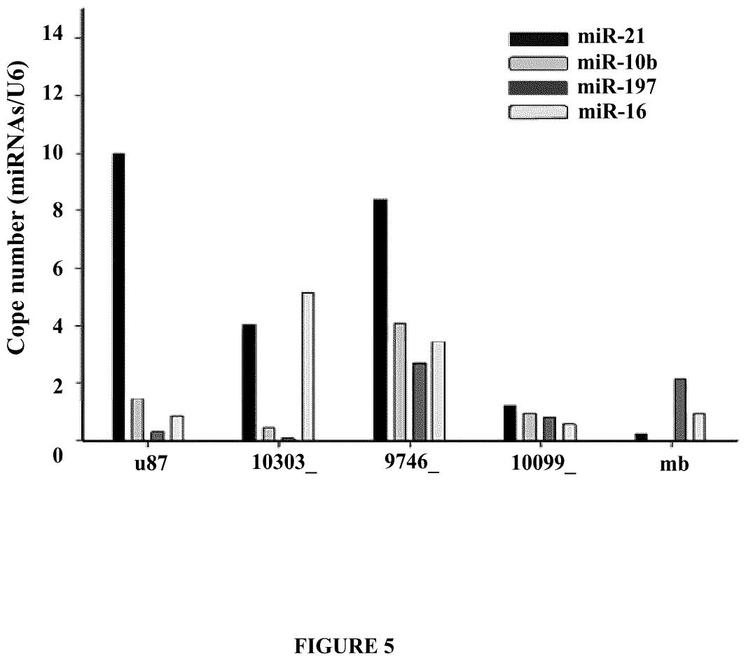

To obtain the copy numbers for each miRNA, the standard curves of miR-21 and U640 were used, and the average standard curve was calculated and used based on the standard curves of miR-31, miR-96, and miR-135b from the same study. According to the average standard curve, the copy numbers of miR-16, miR-10b, and miR-197 were obtained. According to the normalized equation: En=Copy number (target)/Copy number (reference), the relative expression levels of target miRNAs were obtained, as shown in FIG. 5. According to the formula (1), we prepared the qPCR dataset for this validation using the SVM classifier directly. One BVZ sample (10099) was found in those three GBM samples, and the positive rate was 33%. If U87 was counted, the rate was 25%. This is also a successful validation since the number of people can only be an integer and cannot be any decimal of a number.

### Example 9

### Pre-Screening of GBM Patients Before any BVZ Treatment to Prevent any Adverse and/or Aging-Related Side Effects for GBM BVZ-Non-Responsive Patients after BVZ Treatments

### Experimental Datasets

The datasets (GSE79671) of GBM patients with BVZ treatment were downloaded from from gene expression omnibus GEO. A sequencing dataset from GBM tissue before and after BVZ treatment was trimmed with Trimmomatic and mapped to the human genome (hg19). After processing, 15630 genes were obtained and expressed as fragments per kilobase per million reads (FPKM). The data of the same patient before and after BVZ treatment were paired for statistical study.

In the Cancer Genome Atlas (TCGA) pilot study, mRNAs and mRNAs expression profiles of GBM were downloaded from TCGA portal (http://cancergenome.nih.gov/dataortal/), containing 426 glioblastoma samples, which were generated using Affymetrix U133A, Affymetrix Exon 1.0 ST, custom Agilent 244 K, and Agilent miRNA array platforms.

Bioinformatics and statistical methods: Multiple bioinformatics methods were performed, including Venn diagram analysis, STRING (version11), g: Profer (ELIXIR, Tartu, Estonia, https://biit.cs.ut.ee/gprofiler/gost), and gene ontology (GO) and Kyoto encyclopedia of genes and genomes (KEGG). In this study, the software used included MATLAB (R2022a, Math Works Inc., Natick, MA), R-project (version 4.2.2, www.rproject. org), and the statistical methods included Student's t-test and paired t-test.

### Patients' Characteristics

This study analyzed BVZ-related network and DE gene expression using 426 GBM patient profiles from which references with complete gene profiles from microarray or high through the sequence.

### Different Gene Expression Patterns Between GBM BVZ Subtypes Before and After BVZ Treatments

Gene expression patterns were significantly different in GBM BVZ-responsive and nonresponsive patients before and after BVZ treatment. The treatment response was assessed according to the RANO criteria and confirmed on the subsequent follow-up MRI, including complete or partial response (CR+PR). After dividing GBM patients into GBM BVZ-responsive and nonresponsive subtypes, paired t-tests before and after BVZ treatment were performed for these two subtypes. For GBM BVZ-responsive patients following BVZ treatment, 469 genes were significantly expressed based on a paired t-test (p<0.05), in which 391 gene expression levels were increased, 78 gene expression levels were decreased, and histograms based on said expression levels indicate specific expression patterns. Alternatively, for GBM BVZ-non-responsive patients following BVZ treatment, 169 genes were significantly expressed, 60 gene expression levels were increased, and 109 gene expression levels were decreased; their expression histogram show no specific expression regions or patterns unlike the GBM BVZ-responsive patients.

For gene ontology (GO) and KEGG pathway analysis of 469 DE mRNAs from GBM BVZ-responsive patients, 84 transcription factors (TF), 10 biological processes (BP), 5 cellular components (CC), and 3 KEGG pathways crossed the threshold (p=0.01) and were involved in BVZ treatment for GBM BVZ-responsive patients. The VEGF pathway exists as one of the KEGG pathways involved. The most involved part was TF, which may be an unexpected result. In contrast, using the same analysis procedure for 169 DE mRNAs from GBM BVZ-non-responsive patients, no functional pathways crossed the threshold (p=0.05) and were involved in BVZ treatment, suggesting that DE mRNAs expression was not specific.

This result indicated that after BVZ treatment, the gene expression patterns and related functional pathways in GBM BVZ-responsive patients were specific and beneficial to the patients; in contract, in the treatment for GBM BVZ-non-responsive patients, the gene expression patterns and related functional pathways were not specific and may not be beneficial to the patients, suggesting the need for prescreened for BVZ therapy.

### Possible Side Effects for GBM BVZ-Nonresponsive Patients Following BVZ Treatments

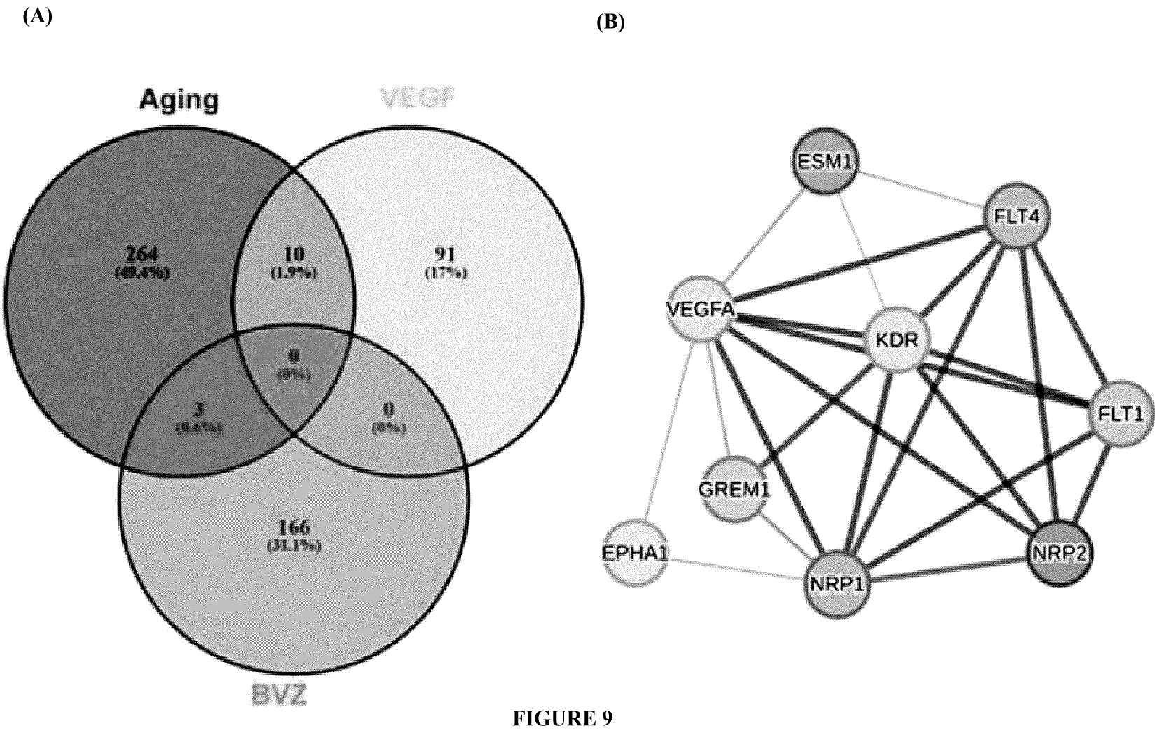

The side effects may be caused when BVZ treats GBM BVZ-non-responsive patients. Several genes were significantly altered in the pathways of apoptosis, inflammation, and cellular proliferation, but they were altered by no more than three genes in the aging pathway. After multiple assessments, significant decreases in Ephrin type A receptor 1 (EPHA1), endothelial cell specific molecular 1 (ESM1), and gremin 1 (GREM1) were found, as shown in FIG. 9A. Compared with pretreatment, the expression levels of EPHA 1 , ESM1, and GREM1 were reduced by 5.25, 5.22, and 2.17 fold (p=0.004, 0.04, and 0.04, n=12) after BVZ treatment, respectively. They were predicted to link to VEGF, but there is no confidence for this connection, and are represented by thin lines rather than thick lines between genes, as shown in FIG. 9B.

Aging-related side effects in GBM BVZ-nonresponsive patients may be caused by BVZ treatment, possibly through significant expression changes inEPHA 1 and ESM1 based on current studies, suggesting the need of prescreening and pre-detection of GBM BVZ-responsive patients for BVZ treatment. An aged animal study showed that after 12 weeks of exercise, marked overexpression of ESM1 was observed in exercised animals compared with control animals, and significantly improved diastolic function was also observed in these animals. After BVZ treatment for GBM BVZ-nonresponsive patients, the expression levels were dramatically decreased, possibly indicating the loss of diastolic function or one of the aging phenotypes. In addition, EphA1 belongs to the family of ephrin receptors that are involved in developmental events, especially in the development of the nervous system. Recent Drosophila studies showed that EphA 1 is an Alzheimer's disease-associated gene, as RNAi mediated knockdown of ephrin significantly impaired fly memory.

Given that patients who received preoperative and postoperative BVZ are known to be at risk of increased morbidity and mortality caused by side effects, patients who do not respond to BVZ treatment should not be taken at risk. From a practical standpoint, patients treated with BVZ are at risk of wound complications including dehiscence, CSF leak, infections, etc. Based on a study of 209 GBM patients, significant healing complications occurred in 44% of patients treated with preoperative BVZ compared to 9% of untreated patients, which significantly increased the rate of morbidity and mortality in this patient population. Careful selection of patients for initiation of BVZ treatment is very important, taking into account laboratory and clinical side effects.

### Example 10

### A New Diagnostic Method for Detection of BVZ-Responsive and Non-Responsive GBM Subtypes Using Multiple Biomarkers and Machine Learning Algorithm Technology

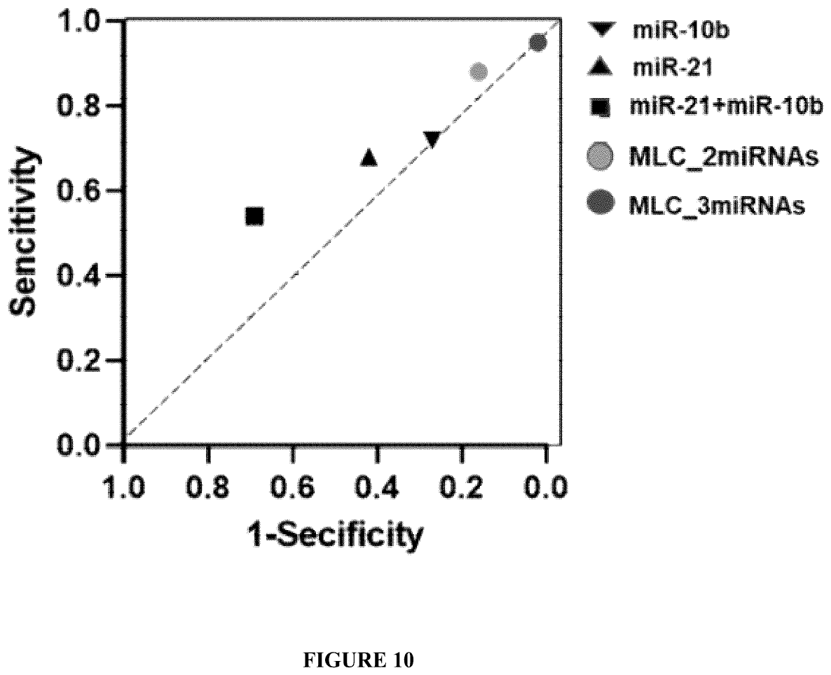

Detection of GBM BVZ-responsive and non-responsive subtypes by a panel of a group of miRNAs used as biomarkers including miR-21, miR-10b, and miR-197 as disclosed herein in the present invention is not possible using traditional methods that do not use artificial intelligence such as machine learning algorithm technology. For example, as shown in the FIG. 10, if miR-21 is used as a biomarker with its threshold to detect the subtypes, the accuracy is about 70%; if miR-10b and its threshold are used to detect the subtypes, the accuracy is about 75%. These accuracies are too low to be used in clinical detection and clinical use. However, when using both miR-21 and miR-10b and their thresholds, as indicated by a black square in the FIG. 10, the accuracy is about 55%, which was lower than that of two biomarkers. Therefore, traditional methods cannot utilize multiple biomarkers for detection.

In this example of the present invention, the three biomarkers including miR-21, miR10b and miR197, were used in combination with a machine learning algorithm, and that led to an accuracy rate of 95%, as shown by a red circle in the FIG. 10.

It will be apparent to those skilled in the art that various modifications and variations can be made in the practice of the present invention without departing from the scope or spirit of the invention. Other embodiments of the invention will be apparent to those skilled in the art from considering of the specification and practice of the invention. It is intended that the specification and examples be considered as exemplary only, with a true scope and spirit of the invention being indicated by the following claims.

### REFERENCES

1. Kim, T. M., Huang, W., Park, R., Park, P. J. & Johnson, M. D. A developmental taxonomy of glioblastoma defined and maintained by MicroRNAs. Cancer Res. 71, 3387-3399. https://doi.org/10.1158/0008-5472. CAN-10-4117 (2011).
2. Diaz, R. J. et al. The role of bevacizumab in the treatment of glioblastoma. J. Neurooncol. 133, 455-467. https://doi.org/10. 1007/s11060-017-2477-x (2017).
3. Batchelor, T. T. et al. AZD2171, a pan-VEGF receptor tyrosine kinase inhibitor, normalizes tumor vasculature and alleviates edema in glioblastoma patients. Cancer Cell 11, 83-95. https://doi.org/10.1016/j.ccr.2006.11.021 (2007).
4. Winkler, F., Osswald, M. & Wick, W. Anti-angiogenics: Their role in the treatment of glioblastoma. Oncol. Res. Treat. 41, 181-186. https://doi.org/10.1159/00048 8258 (2018).
5. Ho, T. K. Random decision forests. In Proceedings of the 3rd International Conference on Document Analysis and Recognition, Montreal, QC 278-282 (1995).
6. McCulloch, W. & Pitts, W. A logical calculus of ideas immanent in nervous activity. Bull. Math. Biophys. 5(4), 115-133. https://doi.org/10.1007/BF024 78259 (1943).
7. Statnikov, A., Aliferis, C. F., Tsamardinos, I., Hardin, D. & Levy, S. A comprehensive evaluation of multicategory classification methods for microarray gene expression cancer diagnosis. Bioinformatics 21, 631-643. https://doi.org/10.1093/bioinformatics/bti033 (2005).
8. Statnikov, A., Wang, L. & Aliferis, C. F. A comprehensive comparison of random forests and support vector machines for microarray-based cancer classification. BMC Bioinform. 9, 319. https://doi.org/10.1186/1471-2105-9-319 (2008).
9. Vapnik, C. C. V. Support-vector networks. Mach. Learn. https://doi.org/10.1007/BF009 94018 (1995).
10. Huang, M. W., Chen, C. W., Lin, W. C., Ke, S. W. & Tsai, C. F. SVM and SVM ensembles in breast cancer prediction. PLoS ONE 12, e0161501. https://doi.org/10.1371/journal.pone.01615 01 (2017).
11. Teplyuk, N. M. et al. MicroRNAs in cerebrospinal fluid identify glioblastoma and metastatic brain cancers and reflect disease activity. Neuro Oncol. 14, 689-700. https://doi.org/10.1093/neuonc/nos074 (2012).
12. Kim, T. M., Huang, W., Park, R., Park, P. J. & Johnson, M. D. A developmental taxonomy of glioblastoma defined and maintained by MicroRNAs. Cancer Res. 71, 3387-3399. https://doi.org/10.1158/0008-5472. CAN-10-4117 (2011).
13. Cancer Genome Atlas Research, N. Comprehensive genomic characterization defines human glioblastoma genes and core pathways. Nature 455, 1061-1068. https://doi.org/10.1038/nature07385 (2008).
14. Siegal, T. et al. Dynamics of circulating hypoxia-mediated miRNAs and tumor response in patients with high-grade glioma treated with bevacizumab. J. Neurosurg. 125, 1008-1015. https://doi.org/10.3171/2015.8.JNS15437 (2016).
15. Shi, J. & Huang, S. W. Predicting and identifying human glioblastoma MiRNA targets using RRSM and qPCR methods. Grant Med. J. 02, 7 (2017).
16. Ahmed, S. P., Castresana, J. S. & Shahi, M. H. Glioblastoma and MiRNAs. Cancers (Basel) https://doi.org/10.3390/cancers13071581 (2021).
17. Tian, L. Q. et al. MicroRNA-197 inhibits cell proliferation by targeting GAB2 in glioblastoma. Mol. Med. Rep. 13, 4279-4288. https://doi.org/10.3892/mmr.2016.5076 (2016).
18. Xin, J. et al. FUS1 acts as a tumor-suppressor gene by upregulating miR-197 in human glioblastoma. Oncol. Rep. 34, 868-876. https://doi.org/10.3892/or.2015.4069 (2015).
19. Cai, J. et al. MicroRNA-542-3p suppresses tumor cell invasion via targeting AKT pathway in human astrocytoma. J. Biol. Chem. 290, 24678-24688. https://doi.org/10.1074/jbc.M115.649004 (2015).
20. Pang, H., Zheng, Y., Zhao, Y., Xiu, X. & Wang, J. miR-590-3p suppresses cancer cell migration, invasion and epithelial-mesenchymal transition in glioblastoma multiforme by targeting ZEB1 and ZEB2. Biochem. Biophys. Res. Commun. 468, 739-745. https://doi.org/10.1016/j.bbrc.2015.11.025 (2015).
21. Chen, F. et al. Up-regulation of microRNA-16 in glioblastoma inhibits the function of endothelial cells and tumor angiogenesis by targeting Bmi-1. Anticancer Agents Med. Chem. 16, 609-620. https://doi.org/10.2174/1871520615666150916092251 (2016).
22. Nichio, B. T. L., Marchaukoski, J. N. & Raittz, R. T. New tools in orthology analysis: A brief review of promising perspectives. Front. Genet. 8, 165. https://doi.org/10.3389/fgene.2017.00165 (2017).
23. Shen, G., Li, X., Jia, Y. F., Piazza, G. A. & Xi, Y. Hypoxia-regulated microRNAs in human cancer. Acta Pharmacol. Sin. 34, 336-341. https://doi.org/10.1038/aps.2012.195 (2013).
24. Shi, J. Predicted regulatory pathways for long noncoding RNA-SNHG7 via miR-34a and its targets in Alzheimer's disease. In IEEE International Conference on Bioinformatics and Biomedicine (IEEE BIBM). https://doi.org/10.1109/BIBM49941.2020.9313260 (2020).
25. Haefeli, J. et al. A data-driven approach for evaluating multi-modal therapy in traumatic brain injury. Sci. Rep. 7, 42474. https://doi.org/10.1038/srep42474 (2017).
26. Shi, J., Parada, L. F. & Kernie, S. G. Bax limits adult neural stem cell persistence through caspase and IP3 receptor activation. Cell Death Differ. 12, 1601-1612. https://doi.org/10.1038/sj.cdd.4401676 (2005).
27. Ciafre, S. A. et al. Extensive modulation of a set of microRNAs in primary glioblastoma. Biochem. Biophys. Res. Commun. 334, 1351-1358. https://doi.org/10.1016/j.bbrc.2005.07.030 (2005).
28. Lambrou, G. I., Zaravinos, A. & Braoudaki, M. Co-deregulated miRNA signatures in childhood central nervous system tumors: In Search for common tumor miRNA-related mechanics. Cancers (Basel) https://doi.org/10.3390/cancers13123028 (2021).
29. Shi, J. Considering exosomal miR-21 as a biomarker for cancer. J Clin Med https://doi.org/10.3390/jcm5040042 (2016).
30. Wu, L. et al. MicroRNA-21 expression is associated with overall survival in patients with glioma. Diagn. Pathol. 8, 200. https://doi.org/10.1186/1746-1596-8-200 (2013).
31. Lardizabal, M. N. et al. Reference genes for real-time PCR quantification of microRNAs and messenger RNAs in rat models of hepatotoxicity. PLoS ONE 7, e36323. https://doi.org/10.1371/journal.pone.0036323 (2012).
32. Morata-Tarifa, C. et al. Validation of suitable normalizers for miR expression patterns analysis covering tumour heterogeneity. Sci. Rep. 7, 39782. https://doi.org/10.1038/srep39782 (2017).
33. MATLAB. version r2021b. Natick, Massachusetts: The MathWorks Inc. (2021).
34. Domingos, P. M. A few useful things to know about machine learning. Commun. ACM 55(10), 78-87. https://doi.org/10.1145/2347736.2347755 (2012).
35. Demsar, J. & Zupan, B. Hands-on training about overfitting. PLoS Comput Biol 17, e1008671. https://doi.org/10.1371/journal.pcbi.1008671 (2021).
36. Wu, Y. et al. Research progress of gliomas in machine learning. Cells https://doi.org/10.3390/cells10113169 (2021).
37. Zhang, W. et al. miR-181d: A predictive glioblastoma biomarker that downregulates MGMT expression. Neuro Oncol. 14, 712-719. https://doi.org/10.1093/neuonc/nos089 (2012).
38. Bolger AM, Lohse M, Usadel B (2014) Trimmomatic: a flexible trimmer for Illumina sequence data. Bioinformatics 30: 2114-20.
39. Urup T, Staunstrup LM, Michaelsen SR, Vitting-Seerup K, Bennedbaek M, et al. (2017) Transcriptional changes induced by bevacizumab combination therapy in responding and non-responding recurrent glioblastoma patients. BMC Cancer 17: 278.
40. Cancer Genome Atlas Research Network (2008) Comprehensive genomic characterization defines human glioblastoma genes and core pathways. Nature 455: 1061 1068.
41. Nichio B T L, Marchaukoski J N, Raittz R T (2017) New Tools in Orthology Analysis: A Brief Review of Promising Perspectives. Front Genet 8: 165.
42. Szklarczyk D, Kirsch R, Koutrouli M, Nastou K, Mehryary F, et al. (2023) The STRING database in 2023: protein-protein association networks and functional enrichment analyses for any sequenced genome of interest. Nucleic Acids Res 51: D638-D646.
43. Shi J (2020) Predicted Regulatory Pathways for Long Noncoding RNA-SNHG7 via miRNA-34a and its Targests in Alzheimer's Disease. IEEE Xplore. IEEE BIBM.
44. Shi J, Huang S W (2017) Predicting and Identifying Human Glioblastoma MiRNA Targets Using RRSM and qPCR Methods. The Grant Medical Journals 02: 7.
45. Shi J (2016) Considering Exosomal miR-21 as a Biomarker for Cancer. J Clin Med 5: 42.
46. Borzsei D, Priksz D, Szabo R, Bombicz M, Karacsonyi Z, et al. (2021) Exercise-mitigated sex-based differences in aging: from genetic alterations to heart performance. Am J Physiol Heart Circ Physiol 320: H854-H866.
47. Buhl E, Kim YA, Parsons T, Thu B, Santa-Maria I, et al. (2022) Effects of Eph/ephrin signalling and human Alzheimer's disease-associated EphAl on Drosophila behaviour and neurophysiology. Neurobiol Dis 170: 105752.
48. Clark A J, Butowski N A, Chang S M, Prados M D, Clarke J, et al. (2011) Impact of bevacizumab chemotherapy on craniotomy wound healing. J Neurosurg 114: 1609-1616.

What is claimed is:

1. A method for classification, clinical detection, and diagnosis of bevacizumab (BVZ)-responsive and non-responsive glioblastoma multiforme (GBM) subtypes referred to collectively as BVZ GBM subtypes based on a panel of a group of micro RNAs (miRNAs) to be used as biomarkers in combination with computer implemented machine learning algorithms for said classification clinical detection, and diagnosis of the BVZ GBM subtypes, the method comprising the steps of:
(i) obtaining the expression z-scores of miRNAs and mRNAs and miRNA expression profiling data for GBM patients including data_expression_merged_median_Zscore and data_expression_miRNA downloaded from the Cancer Genome Atlas (TCGA) pilot study datasets, and downloading additional datasets, including clinicopathological annotations and methylation data for the GBM patients;
(ii) defining, classifying, and selecting the GBM patients in terms of BVZ responsiveness into the BVZ GBM subtypes which are identified and classified as a BVZ-responsive GBM subtype and a BVZ-non-responsive GBM subtype also referred to as control for GBM patient selection;
(iii) assessing, demonstrating, and comparing the obtained BVZ-responsive GBM subtype to the BVZ-non-responsive GBM subtype by analyzing the clinicopathological annotations and methylation data for the GBM patients including survival time, heredity and mutations, and methylation, and the expression z-scores of miRNAs and mRNAs and miRNA expression profiling data for differential expression (DE) of miRNAs and mRNAs, clustering and GO analysis thereof to obtain analysis results applicable for further GBM patient selection and classification into the BVZ GBM subtypes;
(iv) performing data statistical analysis on the DE miRNAs of the step (iii) by performing a t-test on the obtained z-score matrix and comparing the BVZ GBM subtypes to determine which miRNAs differed most between the BVZ-responsive GBM subtype and the BVZ-non-responsive GBM subtype to obtain analyzed DE miRNAs;
(v) ranking the analyzed DE miRNAs of the step (iv) to obtain highly relevant miRNAs for the BVZ GBM subtypes;
(vi) performing hierarchical clustering of DE miRNAs between the BVZ GBM subtypes and then visualizing the clustering as a heatmap using MeV software to visualize the highly relevant miRNAs for each of the BVZ GBM subtypes, including, the BVZ-responsive GBM subtype and the BVZ-non-responsive GBM subtype;
(vii) performing data statistical analysis on the DE mRNAs of the step (iii) by performing a t-test on the obtained z-score matrix and comparing the BVZ GBM subtypes to determine which mRNAs and consequently their coded genes differed most between the BVZ-responsive GBM subtype and the BVZ-non-responsive GBM subtype to identify significant genes for the BVZ GBM subtypes;
(viii) performing hierarchical clustering of the significant genes between the BVZ GBM subtypes and then visualizing the clustering as a heatmap using MeV software to visualize the significant genes in terms of DE mRNAs for each of the BVZ GBM subtypes, including, the BVZ-responsive GBM subtype and the BVZ-non-responsive GBM subtype;
(ix) constructing three machine learning algorithms and comparing the three machine learning algorithms;
(x) preparing, implementing, modifying, and optimizing supervised machine learning classifiers for each of the three machine learning algorithms of the step (ix) based on the defined and classified BVZ GBM subtypes and the matrices obtained based on the steps (ii) to (viii) above based on z-scores for DE of miRNAs, finding the best combination of miRNAs, performing cross-validation, and modifying said classifiers to train and test a first set of clinical datasets;
(xi) constructing further of the supervised machine learning classifiers of the step (x) for each of the three machine learning algorithms of the step (ix) for clinical use based on current clinical techniques without the use of z-scores to classify, detect, and diagnose the BVZ GBM subtypes;
(xii) comparing the constructed supervised machine learning classifiers as constructed in the step (xi) for accuracy rate resulting in selection of the constructed SVM classifier from the step (xi) for further clinical use;
(xiii) optimizing and evaluating further of the constructed SVM classifier from the step (xi) to obtain a new and improved machine learning process of the SVM classifier for best accuracy rate and results in identification of a panel of a group of miRNAs to be used as biomarkers for the classification and clinical detection of the BVZ GBM subtypes obtained as the best combination with a step-by-step optimization and evaluation process for the highly relevant miRNAs of the step (v);
(xiv) performing stratified k-fold cross-validation for the SVM classifier to prevent overfitting;
(xv) obtaining the confusion matrix, sensitivity, specificity, and mean accuracy on which the receiver operating curve (ROC) of the SVM classifier was generated, and the said SVM classifier was used for further validation;
(xvi) validating the SVM classifier of the step (xv) by using a second set of clinical datasets, wherein the defining, classifying, and selecting the GBM patients of the step (ii) in terms of BVZ responsiveness into four subgroups based on the expression levels or z-scores of miR-21 and miR-10b as obtained by using the R program and a Venn diagram method,
wherein among the four subgroups, the patient IDs of the GBM patients with high expression of both miR-21 and miR-10b are identified as a first subgroup, and defined and classified as the BVZ-responsive GBM subtype, while the patient IDs of other three subgroups of the GBM patients, which include a second subgroup with both miR-21 and miR-10b downregulated referred to as DD group, a third subgroup with miR-21 upregulated and miR-10b downregulated referred to as UD group, and a fourth subgroup with miR-21 downregulated and miR-10b upregulated referred to as DU group, are identified, combined, defined, and classified as the BVZ-non-responsive GBM subtype,
wherein the BVZ-responsive GBM subtype are the GBM patients who are highly responsive to BVZ treatment, while the BVZ-non-responsive GBM subtype did not highly respond to BVZ treatment,
wherein the comparing of the three machine learning algorithms in the step (ix) is done using the same dataset and based on the expression z-scores of miR-21 and miR-10b, represented by a batch of miRNA expression datasets, and
wherein the analysis the steps (iii) to (viii) further established the expression changes in levels of miR-21 and miR-10b as important for classification of the GBM patients into distinct subgroups.

2. The method of claim 1, wherein the defining, classifying, and selecting the GBM patients in the step (ii) of the claim 1 is based on the high expression levels of miR-21, and miR-10b defined as z-scores greater than zero, while low levels of miR-10b or miR-21 are defined as z-scores below or zero, and wherein the high expression levels of miR-21, and miR-10b, were negatively correlated and significantly associated with decreased tumor diameters in the BVZ treated group, but not in a temozolomide (TMZ)-treated group based on the data from the datasets of the step (i) of the claim 1.

3. The method of claim 1, wherein the defining, classifying, and selecting the GBM patients in the step (ii) of the claim 1 is based in terms of BVZ responsiveness in the serum, cerebrospinal fluid (CSF), and tumor tissues of the GBM patients as compared with normal control patients based on the data from the datasets of the step (i) of the claim 1.

4. The method of claim 1, wherein the ranking of the analyzed DE miRNA in the step (v) of the claim 1 is done by a p-value with a cutoff at 0.0001 and standard Bonferroni correction for each subtype to obtain the highly relevant miRNAs for the BVZ GBM subtypes that pass the threshold, and said highly relevant miRNAs include miR-10b, miR-140-3p, miR-142-3p, miR-148a, miR-197, miR-21, miR-324-3p, miR-328, miR-424, miR-542-3p, miR-574-3p, miR-590-5p, miR-636, and miR-92a.

5. The method of claim 1, wherein the identifying significant genes in the step (vii) of the claim 1 is performed using Student's t-test at a p-value with a cutoff at 0.01 and with false discovery correction, standard Bonferroni correction referred to as FDC analysis to obtain the significant genes for the BVZ GBM subtypes that pass the threshold between these two subgroups as gene signatures of GBM BVZ subtypes, and said significant genes include annexin A2 (ANXA2), homeobox D10 (HOXD10), ephrin Al (EFNA1), homeobox Dll (HOXD11), annexin A2 pseudogene 2 (ANXA2P2), GREB1 like retinoic acid receptor coactivator (GREB1L), and FKBP prolyl isomerase 9 (FKBP9).

6. The method of claim 1, wherein the assessing, demonstrating, and comparing in the step (iii) of the claim 1 further established differences between the two BVZ GBM subtypes including the BVZ-responsive GBM subtype and the BVZ-non-responsive GBM subtype in terms of differences in survival time, wherein said BVZ-responsive GBM subtype as obtained and classified according to the high expression of miR-21 and miR-10b in GBM patients is identified as a new BVZ subtype of GBM, absent in any of the subclasses based on other miRNA consensus clustering, and wherein the mean survival time of the GBM patients classified as the BVZ-responsive GBM subtype is significantly shorter than that of the BVZ-non-responsive GBM subtype indicating that the BVZ-responsive GBM subtype is more aggressive than the BVZ-non-responsive GBM subtype.

7. The method of claim 1, wherein the three machine learning algorithms in the step (ix) of the claim 1 include support vector machine (SVM), random forest (RF), and neural network (NN), and wherein the respective methods are fitcsvm for SVM, fitctree for RF, and fitcnet for NN.

8. The method of claim 1, wherein the further of the supervised machine learning classifiers of the step (x) of the claim 1 for each of the three machine learning algorithms of the step (ix) for clinical use based on current clinical techniques without the use of z-scores involves selecting miR-16 as the endogenous control, and the use of the following formula 1 for the analysis of expression data for any one of the miRNAs identified as miR-i from a microarray expression dataset calculated to the loge-transformed ratios, and performed as follows:
Ri=log₂[(miR-i)/(miR-16)] Formula 1
where, Ri is the transformed ratio of control miR-16, and miR-i is the expression values of any one of the miRNAs being considered in this analysis for clinical use without the use of z-scores.

9. The method of claim 1, wherein the optimizing and evaluating further of the constructed SVM classifier is done by the use of the sequentialfs function to add and try more variants for better prediction to reach the new and improved machine learning process of the SVM classifier for best accuracy rate obtained herein, wherein the optimizing and evaluating further of feature subsets based on the highly relevant miRNAs of the step (v) of the claim 1 is done using formula 2 as follows:
sequentialfs(fun, X, y) Formula 2
where, said formula 2 selects a subset of features from the data matrix X and sequentially compares features until the best candidate is found to best predict the data in y.

10. The method of claim 1, wherein the step-by-step optimization and evaluation of each possibility and each combination for each of the highly relevant miRNAs of the step (v) of the claim 1 leads to the best combination that can be obtained from this process which is identified as the panel of a group of miRNAs to be used as biomarkers for the classification and clinical detection of the BVZ GBM subtypes.

11. The method of claim 1, wherein the panel of a group of miRNAs to be used as biomarkers for the classification and clinical detection of the BVZ GBM subtypes includes miR-21, miR-10b, and miR-197.

12. The method of claim 1, wherein the stratified k-fold cross-validation is performed using crossvalind and fitcsvm programs.

13. The method of claim 1, wherein the second set of clinical datasets for validating the SVM classifier in the step (xvi) of the claim 1 comprise different miRNA microarray datasets.

14. The method of claim 1, wherein the second set of clinical datasets for validating the SVM classifier in the step (xvi) of the claim 1 comprise real-time quantitative polymerase chain reaction (qPCR) data obtained from total RNA isolated from tissues of GBM patients in molecular experiments.

15. The method of claim 1, wherein the classification, clinical detection, and diagnosis of the BVZ GBM subtypes based combining of multiple miRNA biomarkers in the form of the panel of the group of miRNAs to be used as biomarkers with computer implemented machine learning algorithms achieves an accuracy rate of at least 95%, which is suitable for successful use in clinical detection and application, whereas traditional methods using one or more of said miRNA biomarkers of the panel including miR-21, miR-10b, and miR-197, in any combination thereof, with the respective thresholds achieve an accuracy rate that is too low and unsuitable for use in clinical detection and application for classification, detection, diagnosis, and prediction of the BVZ GBM subtypes.

16. A diagnostic method for detection of bevacizumab (BVZ)-responsive and non-responsive glioblastoma multiforme (GBM) subtypes referred to collectively as BVZ GBM subtypes comprising combining a panel of a group of miRNAs to be used as biomarkers to classify, detect, diagnose, and predict BVZ-responsiveness in GBM tissues obtained from GBM patients with machine learning algorithms, the method comprising the steps of:
(a) obtaining tissues from subjects;
(b) isolating total RNA from the tissues in the step (a);
(c) evaluating the quality and quantity of the isolated total RNA in the step (b);
(d) performing reverse transcription (RT) on the isolated total RNA after its evaluation in the step (c);
(e) performing real-time quantitative polymerase chain reaction (qPCR) amplification of miRNAs after RT of the step (d);
(f) obtaining copy numbers to quantify amplification of selected miRNAs with respect to endogenous control RNAs calculated in terms of Ct values and average standard curves to obtain copy numbers from the qPCR amplification performed in the step (e);
(f) using a normalized equation as follows: En=Copy number (target)/Copy number (reference), wherein, the target is any one of the selected miRNAs from the step (f) and reference is an endogenous control, which is U6 RNA, to obtain the relative expression levels target miRNAs;
(g) using the formula 1 and calculation process as follows:
Ri=log₂[(miR-i)/(miR-16)] Formula 1
to convert the obtained relative expression levels of the target miRNAs referred to as miR-i in the formula 1 and represents any one of the experimental miRNAs of a panel of a group of miRNAs to be used as biomarkers to classify, detect, classify, and predict BVZ-responsiveness in GBM tissues obtained from GBM patients in comparison to the endogenous control miRNA of formula 1, miR-16 into data, where, Ri is the loge-transformed ratio of control miR-16;
(h) combining multiple miRNA biomarkers with machine learning algorithms by using the data obtained in the step (g) as input dataset for a support vector machine (SVM) classifier as obtained in the claim 1 for machine learning algorithms that leads to the classification, detection, diagnosis, and prediction of the BVZ GBM subtypes based on the differential expression (DE) pattern of the experimental miRNAs of the panel of the group of miRNAs to be used as biomarkers in the GBM tissues,
wherein subjects comprise patients with GBM, and malignant glioma,
wherein the tissues comprise serum, cerebrospinal fluid (CSF), and tumor tissues,
wherein the tissues can be fresh or frozen,
wherein the experimental miRNAs in the panel of the group of miRNAs to be used as biomarkers to classify, detect, diagnose, and predict BVZ-responsiveness in GBM tissues obtained from GBM patients comprise miR-21, miR-10b, and miR-197, and
wherein the combining multiple miRNA biomarkers with machine learning algorithms in the step (h) achieves an accuracy rate of at least 95%, which is suitable for successful use in clinical detection and application, whereas traditional methods using one or more said miRNA biomarkers of the panel including miR-21, miR-10b, and miR-197, in any combination thereof, with the respective thresholds achieve an accuracy rate that is too low and unsuitable for use in clinical detection and application for classification, detection, diagnosis, and prediction of the BVZ GBM subtypes.

17. A method for prescreening of patients with glioblastoma multiforme (GBM) referred to as GBM patients to prevent adverse aging-related side effects caused due to treatment and therapy with an anti-angiogenetic treatment, bevacizumab (BVZ) therapy for GBM patients that targets vascular endothelial growth factor A (VEGF) based on bioinformatics analysis, the method comprising the steps of:
(i′) obtaining mRNAs and mRNAs expression profiles for GBM patients downloaded from the Cancer Genome Atlas (TCGA) pilot study datasets, and downloading additional datasets, including clinicopathological annotations and methylation data for the GBM patients, and obtaining other datasets of GBM patients from gene expression omnibus (GEO) with sequencing datasets before and after BVZ treatment comprising expression data of mRNAs for the same patient before and after BVZ treatment;
(ii′) performing analysis using multiple bioinformatics and statistical methods including Venn diagram analysis, STRING (versionll) analysis, g: Profer (ELIXIR, Tartu, Estonia, https://biit.cs.ut.ee/gprofiler/gost) analysis, gene ontology (GO) and Kyoto encyclopedia of genes and genomes (KEGG) analysis, using software including MATLAB, R-project, and the statistical methods including Student's t-test and paired t-test on the datasets for the GBM patients of the step (i′);
(iii′) using the method of the claim 1 for classification of the datasets of the step (i′) into the BVZ GBM subtypes of the claim 1 classified as BVZ-responsive GBM subtype and BVZ-non-responsive GBM subtype;
(iv′) analyzing differential expression (DE) of mRNAs for differential gene expression patterns and BVZ-related networks before and after BVZ treatment as obtained from the analysis in the step (ii′) for the GBM patients classified as the BVZ-responsive GBM subtype and the BVZ-non-responsive GBM subtype based on the step (iii′) to obtain, assess, and analyze a BVZ treatment response;
(v′) performing paired t-tests before and after BVZ treatment on the two BVZ GBM subtypes after classifying and dividing the GBM patients into the BVZ-responsive GBM subtype and the BVZ-non-responsive GBM subtype in the step (iv′) to obtain significantly differentially expressed mRNAs and corresponding genes for each subtype;
(vi′) performing gene ontology (GO) and KEGG pathway analysis of the significantly differentially expressed mRNAs and corresponding genes as obtained in the step (v′) for the GBM patients classified as the BVZ-responsive GBM subtype and the BVZ-non-responsive GBM subtype to obtain gene expression patterns and functional pathways associated with BVZ treatment as obtained in analyzed data after the BVZ treatment;
(vii′) assessing, analyzing, and comparing the results of gene expression patterns and functional pathways obtained in the step (vi′) after the BVZ treatment for the GBM patients classified as the BVZ-responsive GBM subtype and the BVZ-non-responsive GBM subtype;
(viii′) identifying and assessing a relationship between aging-associated genes, VEGF-associated genes, and DE mRNAs before and after BVZ treatment in the BVZ-non-responsive subtype of the GBM patients based on the results of gene expression patterns and functional pathways of the step (vii′) to identify and assess the adverse aging-related side effects caused when BVZ is administered for treatment in the BVZ-non-responsive subtype of the GBM patients;
(ix′) prescreening and pre-detection of the GBM patients to be classified, detected, and diagnosed as the BVZ-non-responsive subtype based on a combination of biomarkers and machine learning algorithms of the claim 1 to prevent said adverse aging-related side effects of the step (viii′) from being caused due to BVZ treatment and therapy in prescreened and pre-detected BVZ-non-responsive subtype of the GBM patients and for careful selection of the GBM patients who are the BVZ-responsive subtype for initiation of BVZ treatment,
wherein the significantly differentially expressed mRNAs and corresponding genes in the step (v′) are obtained using a p-value with a cutoff at 0.05 for each subtype,
wherein the results of gene expression patterns and functional pathways associated with BVZ treatment as obtained in analyzed data after the BVZ treatment in the step (vi′) are obtained using a p-value with a cutoff at 0.01 for the BVZ-responsive GBM subtype,
wherein the results of gene expression patterns and functional pathways associated with BVZ treatment as obtained in analyzed data after the BVZ treatment in the step (vi′) lead to no functional pathways or specific gene expression patterns that crossed a threshold using a p-value with a cutoff at 0.05 for the BVZ-non-responsive GBM subtype,
wherein the results of gene expression patterns and functional pathways associated with BVZ treatment as obtained in analyzed data after the BVZ treatment in the step (vi′) for the BVZ-responsive GBM subtype indicate that after BVZ treatment, the gene expression patterns and related functional pathways in the BVZ-responsive GBM patients are specific and beneficial to the patients, and
wherein the results of gene expression patterns and functional pathways associated with BVZ treatment as obtained in analyzed data after the BVZ treatment in the step (vi′) for the BVZ-non-responsive GBM subtype indicate that after BVZ treatment, the gene expression patterns and related functional pathways in the BVZ-non-responsive GBM patients are not specific and not beneficial to the patients.

18. The method of claim 17, wherein the BVZ treatment response in the step (iv′) of the claim 17 is obtained, assessed, and analyzed according to the RANO criteria and confirmed on the subsequent follow-up MRI including complete or partial response (CR+PR).

19. The method of claim 17, wherein the adverse aging-related side effects include aging, wound complications including dehiscence, CSF leaking, and infections.

20. The method of claim 17, wherein the DE mRNAs before and after BVZ treatment in the BVZ-non-responsive subtype of the GBM patients in the step (viii′) of the claim 17 showed significant expression changes and decrease in the levels of mRNAs and corresponding genes including Ephrin type A receptorl (EPHA1), endothelial cell specific molecular 1 (ESM1), and gremin 1 (GREM1) after BVZ treatment when compared to before BVZ treatment.
